(19) United States
(12) Patent Application Publication
(10) Pub. No.: US 2019/0046162 A1
(43) Pub. Date: Feb. 14, 2019

(54) ULTRASONIC SIGNAL PROCESSOR, ULTRASONIC DIAGNOSTIC DEVICE, AND ULTRASONIC SIGNAL PROCESSING METHOD

(71) Applicant: Konica Minolta, Inc., Tokyo (JP)

(72) Inventor: Mineo TSUSHIMA, Kyoto (JP)

(73) Assignee: Konica Minolta, Inc., Tokyo (JP)

(21) Appl. No.: 16/048,469

(22) Filed: Jul. 30, 2018

(30) Foreign Application Priority Data
Aug. 10, 2017 (JP) 2017-155946

(51) Int. Cl. A61B 8/08; A61B 8/14; A61B 8/00; G10K 11/28

(52) U.S. Cl. CPC A61B 8/5269; A61B 8/145; G10K 11/28; A61B 8/4444; A61B 8/4488

(57) ABSTRACT

An ultrasonic signal processor transmitting/receiving an ultrasonic wave to/from a subject by joining an ultrasonic probe including transducers and an acoustic lens to the subject and generating an acoustic line signal based on a reflected ultrasonic wave, the ultrasonic signal processor includes: a transmitter transmitting a transmission ultrasonic wave into the subject; a receiver generating a reception signal sequence corresponding to each transducer based on the reflected ultrasonic wave from the subject; and a phasing adder phasing and adding the reception signal sequences to generate an acoustic line signal, wherein the phasing adder includes a reception time calculator calculating a reception time to when the reflected ultrasonic wave reaches the transducer from an observation point in the subject, an ultrasonic velocity in the acoustic lens is slower than that in a region in contact with the acoustic lens of the subject, and the reception time calculator calculates the reception time.

The entire disclosure of Japanese patent Application No. 2017-155946, filed on Aug. 10, 2017, is incorporated herein by reference in its entirety.

### BACKGROUND

### Technological Field

The present disclosure relates to an ultrasonic signal processor and an ultrasonic diagnostic device including the ultrasonic signal processor, and in particular, to a reception beamforming processing method in the ultrasonic signal processor.

### Description of the Related Art

An ultrasonic diagnostic device transmits an ultrasonic wave to an inside of a subject by an ultrasonic probe (hereinafter referred to as a “probe”) and receives an ultrasonic reflected wave (echo) caused by a difference in acoustic impedance of subject tissues. Further, the ultrasonic diagnostic device generates an ultrasonic tomographic image showing a structure of an internal tissue of the subject on the basis of an electrical signal obtained by the reception of echo and displays the ultrasonic tomographic image on a monitor (hereinafter referred to as a “display”). The ultrasonic diagnostic device is widely used for morphological diagnosis of a living body because the ultrasonic diagnostic device is less invasive to the subject and can observe a state of internal tissues in real time with the tomographic image and the like.

In the ultrasonic diagnostic device, a method called phasing addition method is typically used as reception beamforming of a signal based on the received reflected ultrasonic wave (for example, Masayasu Itoh and Tsuyoshi Mochizuki, “Ultrasonic diagnostic device”, Corona Publishing Co., Ltd., Aug. 26, 2002 (P42-P45)). More specifically, the ultrasonic diagnostic device receives the reflected ultrasonic wave by a plurality of transducers, and performing reception beamforming by delay processing taking account of a propagation path of the reflected ultrasonic wave. With the processing, a spatial resolution and a signal S/N ratio of an obtained acoustic line signal can be improved.

Meanwhile, the ultrasonic probe is provided with an acoustic lens between each transducer and the subject. Since the acoustic lens has a different sound velocity from the subject, refraction of the ultrasonic wave occurs at the interface between the acoustic lens and the subject. Therefore, it is necessary to specify the propagation path of the reflected ultrasonic wave and to perform the reception beamforming in consideration of the presence of the acoustic lens. Although the propagation path of the reflected ultrasonic wave can be specified by using the Pythagorean theorem and the Snell's law, the operation amount is large and thus a technique of applying a correction value by preliminary calculation is conventionally used (see, for example, IP 2017-547 A).

However, in the technique of applying the correction value by preliminary calculation to a delay amount based on the assumption of no-presence of the acoustic lens, the precision of the reception beamforming depends on the amount of correction value data. This is because the degree of influence by an acoustic lens differs depending on a relative positional relationship between an observation point and a reception transducer, and thus one correction value cannot be applied to all of observation points and reception transducers. That is, to improve the density of the observation points and to improve the precision of the correction value, a larger amount of correction value data corresponding to the relative positional relationship between a larger number of observation points and reception transducers is required. Therefore, the precision of the reception beamforming is not improved in the case where the amount of correction value data is small whereas a larger amount of correction value data is required to improve the precision of the reception beamforming. That is, the amount of correction value data and the precision of the reception beamforming have a trade-off relationship.

### SUMMARY

The present invention has been made in view of the above problems, and an objective is to provide an ultrasonic signal processor that performs reception beamforming capable of performing acoustic lens correction with higher precision, and an ultrasonic diagnostic device using the ultrasonic signal processor.

To achieve the abovementioned object, according to an aspect of the present invention, there is provided an ultrasonic signal processor that transmits and receives an ultrasonic wave to and from a subject by joining an ultrasonic probe including a plurality of transducers and an acoustic lens to the subject and generates an acoustic line signal on the basis of a reflected ultrasonic wave, and the ultrasonic signal processor reflecting one aspect of the present invention comprises: a transmitter that transmits a transmission ultrasonic wave into the subject, using the ultrasonic probe; a receiver that generates a reception signal sequence corresponding to each transducer on the basis of the reflected ultrasonic wave from the subject received by the ultrasonic probe; and a phasing adder that phases and adds, with respect to a plurality of observation points in the subject, the reception signal sequences to generate an acoustic line signal, wherein the phasing adder includes a reception time calculator that calculates, for each observation point and for each transducer, a reception time to when the reflected ultrasonic wave reaches the transducer from the observation point, an ultrasonic velocity in the acoustic lens is slower than an ultrasonic velocity in a region of the subject, the region being in contact with the acoustic lens, and the reception time calculator calculates the reception time to when the ultrasonic wave is propagated from the observation point to the transducer, using a maximum refraction point most adjacent to the transducer on a refractive surface that is a boundary surface between the acoustic lens and the subject.

### BRIEF DESCRIPTION OF THE DRAWINGS

The advantages and features provided by one or more embodiments of the invention will become more fully understood from the detailed description given hereinbelow and the appended drawings which are given by way of illustration only, and thus are not intended as a definition of the limits of the present invention:

FIG. 1 is a functional block diagram illustrating a configuration of an ultrasonic diagnostic device according to a first embodiment;

FIGS. 2A and 2B are schematic views of a probe according to the first embodiment;

FIG. 3A is a schematic sectional diagram illustrating phasing addition processing;

FIG. 3B is a schematic sectional diagram illustrating an influence by an acoustic lens;

FIG. 4 is a functional block diagram illustrating a configuration of a reception beamformer according to the first embodiment;

FIG. 5 is a functional block diagram illustrating a configuration of a phasing adder according to the first embodiment;

FIGS. 6A and 6B are schematic diagrams illustrating a propagation path of a reflected ultrasonic wave according to the first embodiment;

FIGS. 7A to 7D are schematic diagrams illustrating processing of searching for a refraction point according to the first embodiment;

FIG. 8 is a flowchart illustrating an acoustic line signal generation operation of the reception beamformer according to the first embodiment;

FIG. 9 is a flowchart illustrating a reception time calculation operation of a reception time calculator according to the first embodiment;

FIGS. 10A and 10B are schematic diagrams illustrating a propagation path of a reflected ultrasonic wave according to a first modification:

FIG. 11 is a schematic diagram for describing processing of searching for a refraction point according to a second embodiment;

FIG. 12 is a flowchart illustrating a reception time calculation operation of a reception time calculator according to the second embodiment;

FIGS. 13A and 13B are schematic diagrams illustrating a propagation path of a reflected ultrasonic wave according to a third embodiment; and

FIGS. 14A and 14B are ultrasonic images according to an example and modifications.

### DETAILED DESCRIPTION OF EMBODIMENTS

Hereinafter, one or more embodiments of the present invention will be described with reference to the drawings. However, the scope of the invention is not limited to the disclosed embodiments.

<<Background to Reach Mode for Carrying Out the Invention>>

The inventor has conducted various studies to improve the precision of reception beamforming without significantly increasing an operation amount.

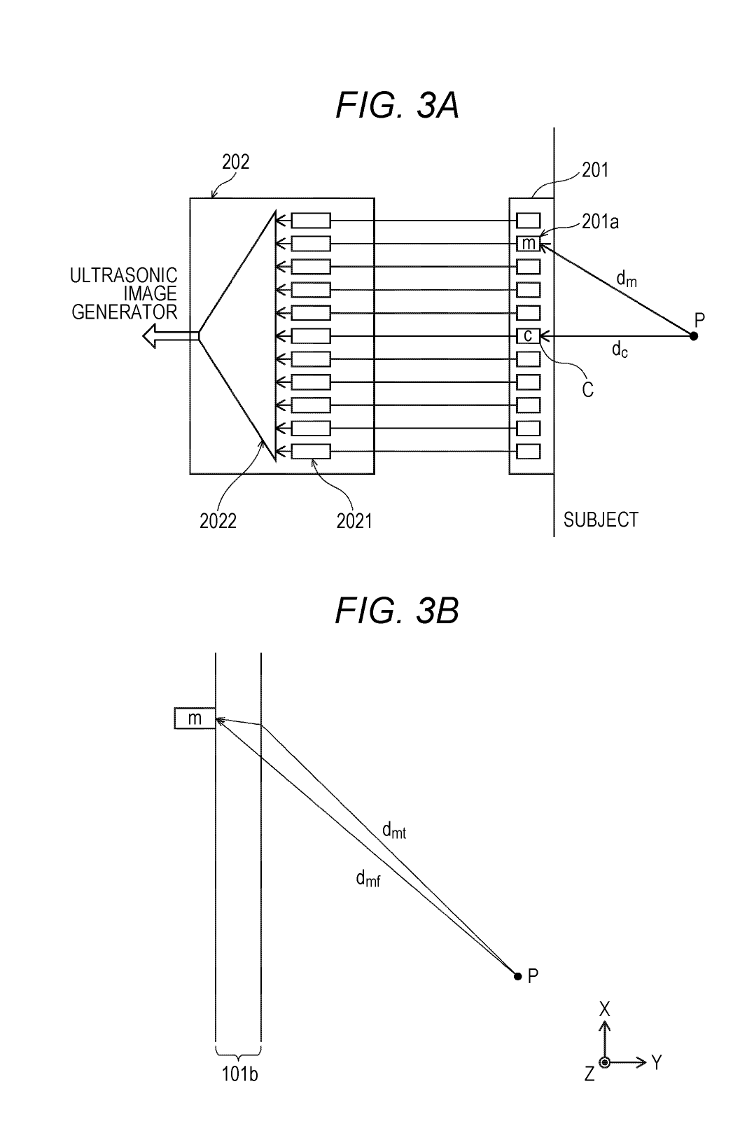

In the phasing addition method, a reflected ultrasonic wave from an observation point P is received by a plurality of reception transducers and a reception signal sequence is generated, and delay processing is performed so that phases of signals based on the reflected ultrasonic wave from the observation point P are matched and synthesis is performed, whereby an S/N ratio is improved. FIG. 3A is a schematic sectional diagram illustrating a principle of phasing addition. As illustrated in FIG. 3A, the reflected ultrasonic wave from the observation point P is received by a plurality of reception transducers. Then, addition is performed after the delay processing is performed by a delayer, and an acoustic line signal is generated. Here, in the delay processing, processing based on a distance between the observation point and the reception transducer is performed. For example, when the distance between the observation point P and the reception transducer C is d_{c}, the distance between the observation point P and the reception transducer M is dₘ, and the ultrasonic velocity in a subject is v, the time to when the reflected wave from the observation point P reaches the reception transducer M is delayed by (dₘ−d_{c})/v from the time to when the reflected wave from the observation point P reaches the reception transducer C. Therefore, the acoustic line signal based on the reflected wave from the observation point P can be generated by performing the delay processing of cancelling a difference in arrival time between the reception transducers with respect to the reflected wave from the same observation point P.

Meanwhile, as described above, since an acoustic lens has a different sound velocity from the subject, it affects a propagation path of the reflected ultrasonic wave. Typically, the acoustic lens is a cylindrical lens in which an aligning direction of the transducers is an axial direction, and has high refractive index (the sound velocity is slow) with respect to the subject because the acoustic lens functions as a lens. The acoustic lens is a plate with a constant thickness in the alignment direction of the transducers. Therefore, in a case where a propagation direction of the ultrasonic wave is not orthogonal to a surface of the acoustic lens, the propagation direction of the ultrasonic wave is changed on an interface between the acoustic lens and the subject by reflection. FIG. 3B is a schematic sectional diagram illustrating the propagation path of the ultrasonic wave in the case where the acoustic lens is present. As illustrated in FIG. 3B, in the case where a path d_{mf} along a straight line connecting the observation point P and the reception transducer m is not orthogonal to the surface of the acoustic lens, the actual propagation path of the ultrasonic wave travels along a path dₘₜ. Generally, since the sound velocity in the acoustic lens is slower than the sound velocity in the subject, the time required for the ultrasonic wave to actually travel along the path dₘₜ is slower than the time required for the ultrasonic wave to travel along the path d_{mf} in the subject at an ultrasonic velocity. Therefore, in the case where the phasing addition is performed without considering the acoustic lens, a calculated delay time and an actual difference in arrival time of the ultrasonic wave between the reception transducers do not coincide. Therefore, even if the delay processing is performed for a plurality of signals based on the reflected ultrasonic wave from the observation point P, the reception times and the phases of the signals do not sufficiently accord, and the S/N ratio is deteriorated and a so-called “out-of-focus” state occurs.

Meanwhile, calculation of the reception time in consideration of the acoustic lens needs to be performed for each observation point and for each transducer. Therefore, there is a known problem that the operation amount is large. In view of the above problem. JP 2017-547 A uses the technique of applying a correction value by preliminary calculation to a delay amount based on the assumption of no-presence of the acoustic lens. However, since the influence by the acoustic lens differs depending on a relative positional relationship between the observation point and the reception transducer, a database of enormous correction values is required to apply accurate correction values to all the observation points and the transducers. That is, there is a trade-off relationship between the precision of acoustic lens correction and a database capacity.

Therefore, the inventor has sought a method of improving the precision of the reception beamforming without significantly increasing the operation amount, has examined a method of calculating the reception time for each observation point and for each transducer by a low-load operation, and has reached the aspect of the present disclosure.

Hereinafter, an ultrasonic image processing method according to an embodiment and an ultrasonic diagnostic device using the method will be described in detail with reference to the drawings.

### First Embodiment

Hereinafter, an ultrasonic diagnostic device 100 according to the first embodiment will be described with reference to the drawings.

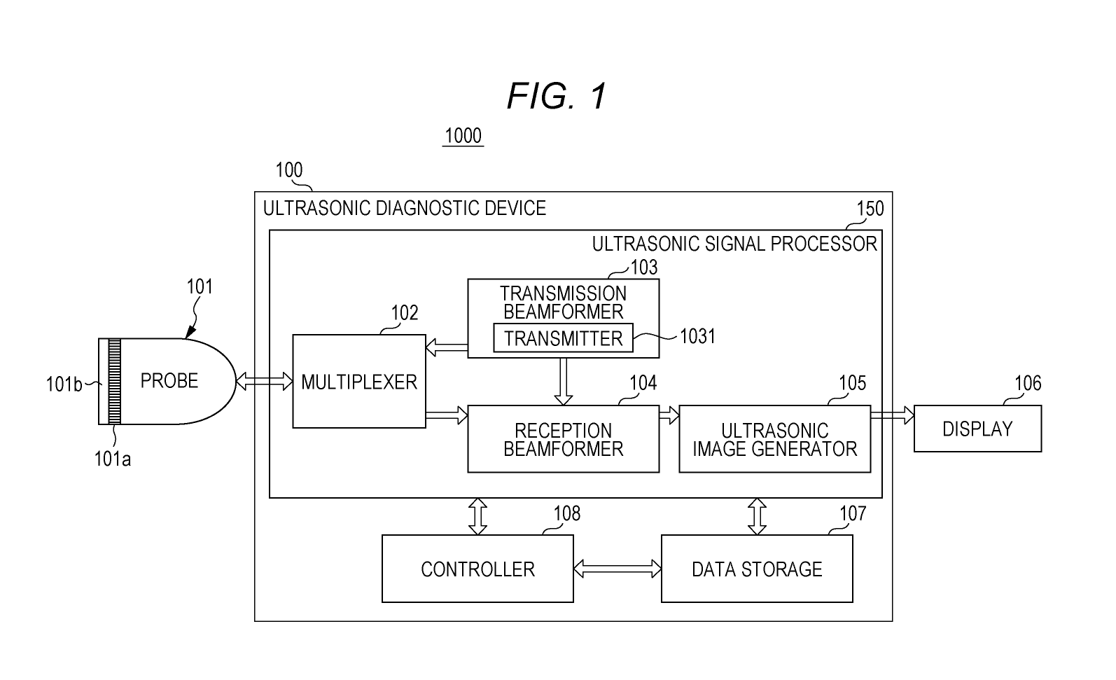

FIG. 1 is a functional block diagram illustrating an ultrasonic diagnostic system 1000 according to the first embodiment. As illustrated in FIG. 1, the ultrasonic diagnostic system 1000 includes a probe 101 including a plurality of transducers 101a that transmits an ultrasonic wave toward a subject and receives a reflected wave, the ultrasonic diagnostic device 100 that causes the probe 101 to transmit and receive the ultrasonic wave and generates an ultrasonic image on the basis of an output signal from the probe 101, and a display 106 that displays the ultrasonic image on a screen. The probe 101 and the display 106 are connectable to the ultrasonic diagnostic device 100. FIG. 1 illustrates a state in which the probe 101 and the display 106 are connected to the ultrasonic diagnostic device 100. Note that the probe 101 and the display 106 may be provided inside the ultrasonic diagnostic device 100.

<Configuration of Ultrasonic Diagnostic Device 100>

The ultrasonic diagnostic device 100 includes a multiplexer 102 that selects each transducer 101a, which is used in transmission or reception, of the plurality of transducers 101a of the probe 101, and secures input and output to the selected transducer 101a, a transmission beamformer 103 that controls timing to apply a high voltage to each transducer 101a of the probe 101 in order to transmit the ultrasonic wave, and a reception beamformer 104 that amplifies electrical signals obtained in the plurality of transducers 101a, applies A/D conversion, and performs reception beamforming to generate an acoustic line signal, on the basis of the reflected wave of the ultrasonic wave received in the probe 101. Further, the ultrasonic diagnostic device 100 includes an ultrasonic image generator 105 that generates an ultrasonic image (B-mode image) on the basis of an output signal from the reception beamformer 104, a data storage 107 that stores the acoustic line signal output by the reception beamformer 104 and the ultrasonic image output by the ultrasonic image generator 105, and a controller 108 that controls the constituent elements.

Among the aforementioned units, the multiplexer 102, the transmission beamformer 103, the reception beamformer 104, and the ultrasonic image generator 105 constitute an ultrasonic signal processor 150.

Each of the elements constituting the ultrasonic diagnostic device 100, for example, the multiplexer 102, the transmission beamformer 103, the reception beamformer 104, the ultrasonic image generator 105, or the controller 108 is realized by a hardware circuit such as a field programmable gate array (FPGA) or an application specific integrated circuit (ASIC). Alternatively, the constituent elements may be realized by software and a programmable device such as a processor. As a processor, a central processing unit (CPU) or a GPGPU can be used, and a configuration using a GPU is called general-purpose computing on graphics processing unit (GPGPU). These constituent elements can be a single circuit part or an aggregate of a plurality of circuit parts. Further, a plurality of constituent elements can be combined into a single circuit part, or a plurality of circuit parts can be formed as an aggregate.

The data storage 107 is a computer-readable recording medium, and for example, a flexible disk, a hard disk, an MO, a DVD, a DVD-RAM, a BD, a semiconductor memory, or the like can be used. Further, the data storage 107 may be a storage device externally connected to the ultrasonic diagnostic device 100.

Note that the ultrasonic diagnostic device 100 according to the present embodiment is not limited to the ultrasonic diagnostic device having the configuration illustrated in FIG. 1. For example, there may be no multiplexer 102, and the transmission beamformer 103 and the reception beamformer 104 may be directly connected to the transducers 101a of the probe 101. Further, the transmission beamformer 103, the reception beamformer 104, or a part thereof may be built in the probe 101. This is not limited to the ultrasonic diagnostic device 100 according to the present embodiment, and the same applies to other ultrasonic diagnostic devices according to other embodiments and modifications described below.

<Configuration of Probe 101>

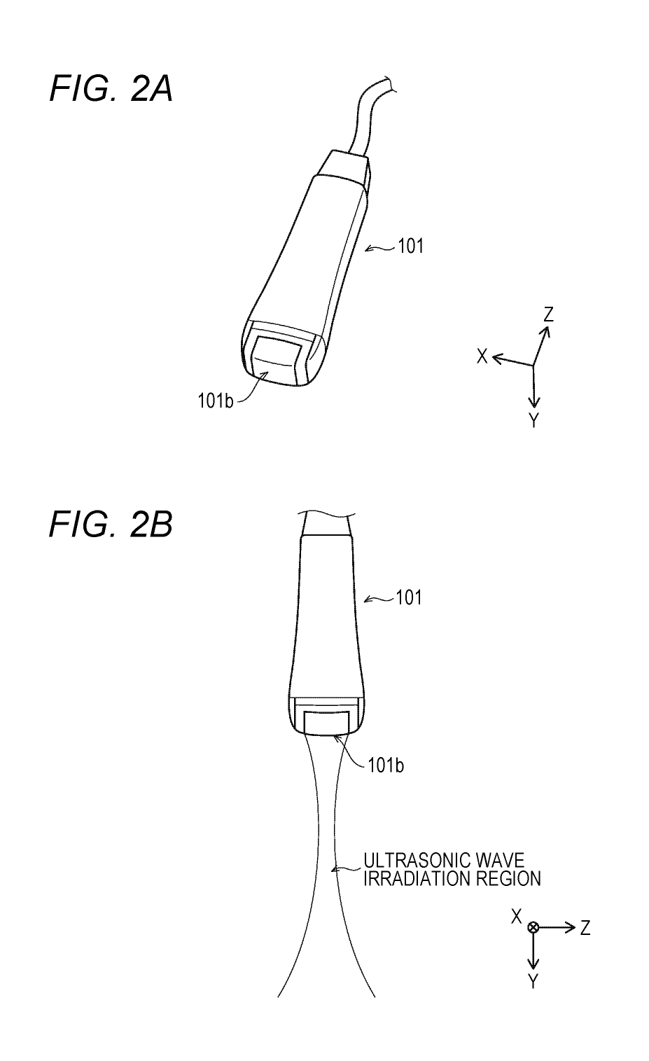

FIG. 2A is an external view of the probe 101. The probe 101 includes the plurality of transducers 101a arrayed in a one-dimensional direction (x direction in the drawing) and an acoustic lens 101b. That is, the probe 101 is a linear probe in which the plurality of transducers 101a is linearly arranged.

Each of the transducers 101a is a piezoelectric element having a function to convert a drive signal supplied from the transmission beamformer 103 via the multiplexer 102 into an ultrasonic wave, convert the received ultrasonic wave into an electrical signal, and output the converted signal to the reception beamformer 104 via the multiplexer 102.

The acoustic lens 101b is a lens for performing transmission and reception beamforming in a direction orthogonal to the arrangement direction of the transducers 101a (z direction in the drawing). Specifically, the acoustic lens 101b is a cylindrical lens made of a material having a smaller sound velocity than a sound velocity of a surface of the subject (that is, a material having a high specific refractive index to the surface of the subject), and having an x axis as an axial direction. With the configuration, as illustrated in FIG. 2B, the ultrasonic wave transmitted from the transducer 101a is not diffused and becomes a beam that is focused to some extent in a yz plane. Although not illustrated in FIG. 2B, in the reception beamforming, the reflected ultrasonic wave from an observation point in an irradiation region of the ultrasonic wave can be received on the yz plane.

<Configuration of Main Part of Ultrasonic Diagnostic Device 100>

The ultrasonic diagnostic device 100 according to the first embodiment has characterized in the transmission beamformer 103 that causes the transducers 101a of the probe 101 to transmit the ultrasonic wave and the reception beamformer 104 that calculates the electrical signals obtained from reception of the ultrasonic reflected wave in the probe 101 to generate the acoustic line signal for generating the ultrasonic image. Therefore, in the present specification, configurations and functions of the transmission beamformer 103 and the reception beamformer 104 will be mainly described. Note that configurations other than the transmission beamformer 103 and the reception beamformer 104 can be the same as those used in known ultrasonic diagnostic devices, and the beamformers according to the present embodiment can be replaced with and used as beamformers of a known ultrasonic diagnostic device.

Hereinafter, configurations of the transmission beamformer 103 and the reception beamformer 104 will be described.

1. Transmission Beamformer 103

The transmission beamformer 103 is connected to the probe 101 via the multiplexer 102, and controls timing of application of a high voltage to each of the plurality of transducers included in a transmission aperture Tx made of a transmission transducer sequence corresponding to a part or all of the plurality of transducers 101a present in the probe 101 for transmitting the ultrasonic wave from the probe 101. The transmission beamformer 103 is configured by a transmitter 1031.

The transmitter 1031 performs transmission processing of supplying a pulse-like transmission signal for causing each transducer included in the transmission aperture Tx to transmit an ultrasonic beam, of the plurality of transducers 101a present in the probe 101, on the basis of a transmission control signal from the controller 108. Specifically, the transmitter 1031 includes, for example, a clock generation circuit, a pulse generation circuit, and a delay circuit. The clock generation circuit is a circuit for generating a clock signal for determining transmission timing of the ultrasonic beam. The pulse generation circuit is a circuit for generating a pulse signal for driving each transducer. The delay circuit is a circuit for setting a delay time of the transmission timing of the ultrasonic beam for each transducer, and performing transmission beamforming of the ultrasonic beam by delaying transmission of the ultrasonic beam by the delay time to form a wavefront of a desired shape. For example, as the number of transducers constituting the transmission aperture Tx, 20 to 100 can be selected where the total number of transducers 101a present in the probe 101 is 192.

The transmission beamformer 103 controls the transmission timing of each transducer such that the transmission timing is further delayed for the transducer located closer to the center of the transmission aperture Tx. With the control, the wavefront of the ultrasonic transmission wave transmitted from the transducer sequence in the transmission aperture Tx is focused at a certain point, that is, a transmission focal point F, in a certain focal depth of the subject. The focal depth of the transmission focal point F can be arbitrarily set. The wavefront focused at the transmission focal point F is diffused again, and the ultrasonic transmission wave is propagated in an hourglass-shaped space sectioned by two intersecting straight lines with the transmission aperture Tx as a base and the transmission focal point F as a constricted part. That is, the ultrasonic wave radiated at the transmission aperture Tx gradually reduces the width (x direction) in the space, minimizes the width at the transmission focal point F, and is diffused and propagated while increasing the width again as proceeding deeper (y direction) than the transmission focal point F. This hourglass-shaped region is an ultrasonic main irradiation region.

Alternatively, for example, in the transmission beamformer 103, the transmission timing of each transducer may be controlled such that the transmission timings of all the transducers in the transmission aperture Tx are caused to accord with one another. Alternatively, for example, in the transmission beamformer 103, the transmission timing of each transducer may be controlled such that a difference in the transmission timing between adjacent transducers becomes constant. With the control, the ultrasonic transmission wave transmitted from the transducer in the transmission aperture Tx becomes a plane wave in which the wavefront is a straight line with a fixed inclination angle (may be 0 degrees) with respect to the x direction. Therefore, the ultrasonic main irradiation region becomes a rectangular or parallelogram region with the transmission aperture Tx as one side.

2. Configuration of Reception Beamformer 104

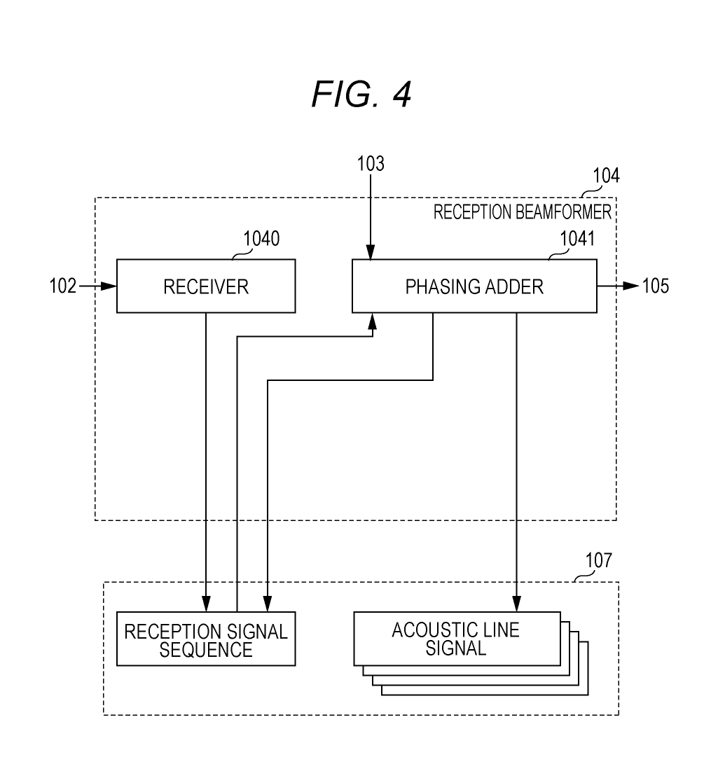

The reception beamformer 104 generates the acoustic line signal from the electrical signal obtained from the plurality of transducers 101a on the basis of the reflected wave of the ultrasonic wave received in the probe 101. Note that the “acoustic line signal” is a signal after phasing addition processing has been performed for a certain observation point. The phasing addition processing will be described below. FIG. 4 is a functional block diagram illustrating a configuration of the reception beamformer 104. As illustrated in FIG. 4, the reception beamformer 104 includes a receiver 1040 and a phasing adder 1041.

Hereinafter, a configuration of each unit constituting the reception beamformer 104 will be described.

(1) Receiver 1040

The receiver 1040 is a circuit connected with the probe 101 via the multiplexer 102, which generates a reception signal (RF signal) obtained by amplifying the electrical signal obtained from the reception of the ultrasonic reflected wave of the probe 101 and then applying AD conversion to the signal in synchronization with a transmission event. The receiver 1040 chronologically generates the reception signal in order of the transmission event, outputs the reception signal to the data storage 107, and stores the reception signal in the data storage 107.

Here, the reception signal (RF signal) is a digital signal obtained by applying A/D conversion to the electrical signal converted from the reflected ultrasonic wave received in each transducer, and forms a sequence of signals continuing in the transmission direction of the ultrasonic wave received in each transducer (in a depth direction of the subject).

As described above, the transmitter 1031 causes each of the plurality of transducers included in the transmission aperture Tx, of the plurality of transducers 101a present in the probe 101, to transmit the ultrasonic beam. In contrast, the receiver 1040 generates a sequence of the reception signals to each transducer on the basis of the reflected ultrasonic waves obtained by respective transducers corresponding to a part or all of the plurality of transducers 101a present in the probe 101 in synchronization with the transmission of the ultrasonic beam. Here, the transducer that receives the reflected ultrasonic wave is called “reception wave transducer”. The number of the reception wave transducers is favorably larger than the number of transducers included in the transmission aperture Tx. Further, the number of the reception wave transducers may be the total number of the transducers 101a present in the probe 101.

(2) Phasing Adder 1041

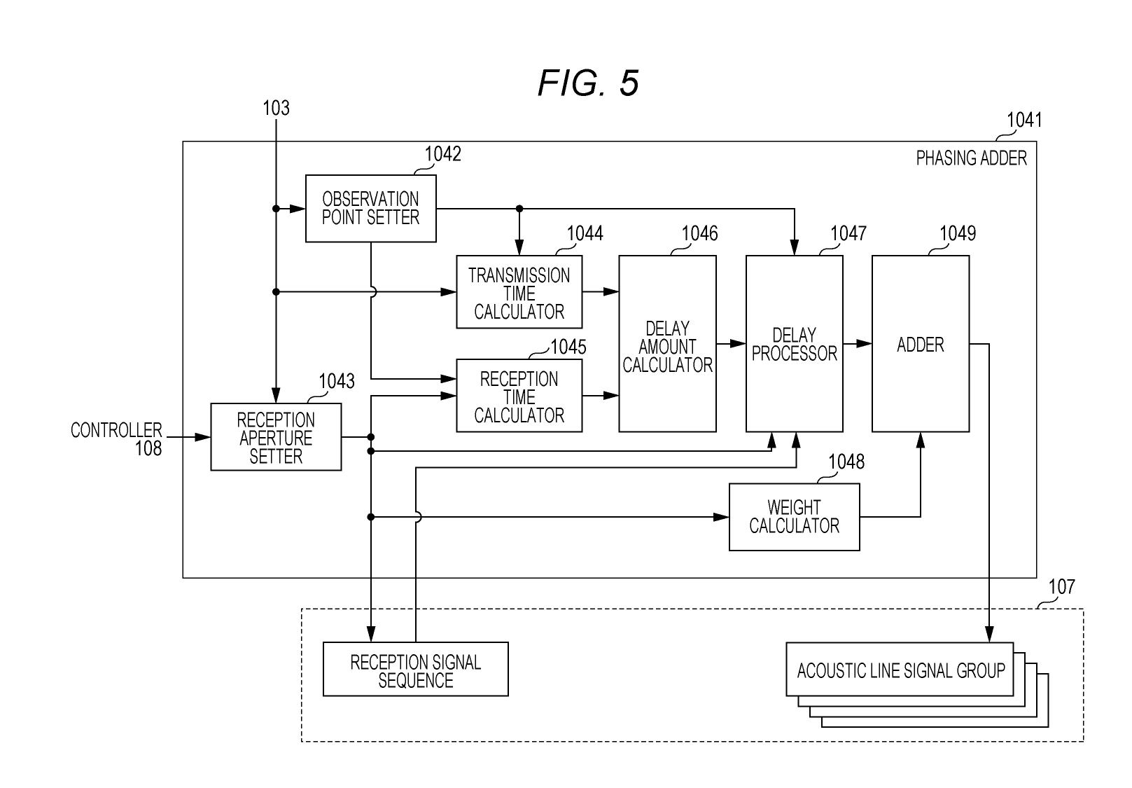

The phasing adder 1041 sets a plurality of observation points Pij for generating a subframe acoustic line signal in the subject in synchronization with the transmission of the ultrasonic beam. Next, the phasing adder 1041 phases and adds, for each of the observation points Pij, the reception signal sequence received by each reception transducer Rk from the observation point. Then, the phasing adder 1041 generates the acoustic line signal in each observation point. FIG. 5 is a functional block diagram illustrating a configuration of the phasing adder 1041. As illustrated in FIG. 5, the phasing adder 1041 includes an observation point setter 1042, a reception aperture setter 1043, a transmission time calculator 1044, a reception time calculator 1045, a delay amount calculator 1046, a delay processor 1047, a weight calculator 1048, and an adder 1049.

Hereinafter, a configuration of each unit constituting the phasing adder 1041 will be described.

i) Observation Point Setter 1042

The observation point setter 1042 sets the plurality of observation points Pij that is targets for generating the acoustic line signal in the subject. The observation point Pij is set for convenience of calculation in synchronization with the transmission of the ultrasonic beam, as an observation target point where generation of the acoustic line signal is performed.

Here, an “acoustic line signal group” is a set of acoustic line signals for all the observation points Pij set in synchronization with the transmission of the ultrasonic beam. That is, the acoustic line signal group is a unit to form a group of signals corresponding to the observation points Pij, which are obtained by one-time transmission of ultrasonic beam and reception processing accompanying the transmission. Note that the acoustic line signals for one frame of the ultrasonic diagnostic device 100 may be composed of one acoustic line signal group or a plurality of acoustic line signal groups.

The observation point setter 1042 sets a plurality of observation points Pij on the basis of information indicating the position of the transmission aperture Tx acquired from the transmission beamformer 103 in synchronization with the transmission of the ultrasonic beam. More specifically, the observation point setter 1042 sets the plurality of observation points Pij in the ultrasonic main irradiation region specified from the position of the transmission aperture Tx.

The set observation points Pij are output to the transmission time calculator 1044, the reception time calculator 1045, and the delay processor 1047.

ii) Reception Aperture Setter 1043

The reception aperture setter 1043 is a circuit for setting a transducer sequence (reception transducer sequence) of a part or all of the plurality of transducers present in the probe 101 as reception transducers to set a reception aperture Rx on the basis of the control signal from the controller 108, and the information indicating the position of the transmission aperture Tx from the transmission beamformer 103.

The reception aperture Rx can be selected such that a sequence center coincides with the transducer spatially most adjacent to the observation point Pij (observation point synchronous type), for example. In this case, the reception aperture Rx is set for each observation point Pij. Alternatively, for example, the reception aperture Rx may be set such that the sequence center of the transmission aperture Tx and the sequence center of the reception aperture Rx coincide with each other (transmission aperture synchronous type). In this case, the reception aperture Rx is set in synchronization with the transmission of the ultrasonic beam.

In any case, to receive the reflected wave from the entire ultrasonic main irradiation region, the number of transducers included in the reception aperture Rx is favorably set to be equal to or larger than the number of transducers included in the transmission aperture Tx in the corresponding transmission event. The number of transducer sequences constituting the reception aperture Rx may be 32, 64, 96, 128, or 192, for example.

Information indicating the position of the selected reception aperture Rx is output to the data storage 107 via the controller 108.

The data storage 107 outputs the information indicating the position of the reception aperture Rx and the reception signal corresponding to the reception transducer to the transmission time calculator 1044, the reception time calculator 1045, the delay processor 1047, and the weight calculator 1048.

iii) Transmission Time Calculator 1044

The transmission time calculator 1044 is a circuit for calculating a transmission time to when the transmitted ultrasonic wave reaches each of the observation points Pij in the subject. The transmission time calculator 1044 calculates, for each of the observation points Pij, the transmission time to when the transmitted ultrasonic wave reaches the observation point Pij in the subject on the basis of the information indicating the position of the transducer included in the transmission aperture Tx acquired from the data storage 107 and the information indicating the position of the observation point Pij acquired from the observation point setter 1042. The transmission time calculator 1044 calculates the transmission time on the basis of, for example, a geometrically calculated distance between the transmission aperture Tx and the observation point Pij.

The transmission time calculator 1044 calculates, for all the observation points Pij, the transmission times to when the transmitted ultrasonic wave reaches the observation points Pij in the subjects in synchronization with the transmission of the ultrasonic beam, and outputs the calculated transmission times to the delay amount calculator 1046.

iv) Reception Time Calculator 1045

The reception time calculator 1045 is a circuit for calculating a reception time to when the reflected wave from the observation point Pij reaches each of the reception transducers Rk included in the reception aperture Rx. The reception time calculator 1045 calculates the reception time to when the transmitted ultrasonic wave is reflected at the observation point Pij in the subject and reaches each reception transducer Rk of the reception aperture Rx on the basis of the information indicating the position of the reception transducer Rk acquired from the data storage 107 and the information indicating the position of the observation point Pij acquired from the observation point setter 1042 in synchronization with the transmission of the ultrasonic beam. Details will be described below.

The reception time calculator 1045 calculates, for all the observation points Pij, the reception times to when the transmitted ultrasonic wave is reflected at the observation points Pij and reaches each reception transducer Rk in synchronization with the transmission of the ultrasonic beam, and outputs the calculated reception times to the delay amount calculator 1046.

v) Delay Amount Calculator 1046

The delay amount calculator 1046 is a circuit for calculating a total propagation time to each reception transducer Ri in the reception aperture Rx from the transmission time and the reception time, and calculating a delay amount to be applied to the reception signal sequence for each reception transducer Rk on the basis of the total propagation time. The delay amount calculator 1046 acquires the transmission time to when the ultrasonic wave transmitted from the transmission time calculator 1044 reaches the observation point Pij, and the reception time to when the ultrasonic wave is reflected at the observation point Pij and reaches each reception transducer Rk. Then, the delay amount calculator 1046 calculates the total propagation time to when the transmitted ultrasonic wave reaches each reception transducer Rk, and calculates the delay amount for each reception transducer Rk according to the difference in the total propagation time for each reception transducer Rk. The delay amount calculator 1046 calculates, for all the observation points Pij, the delay amounts to be applied to the reception signal sequence for each reception transducer Rk, and outputs the delay amounts to the delay processor 1047.

vi) Delay Processor 1047

The delay processor 1047 is a circuit for identifying a reception signal corresponding to the delay amount for each reception transducer Rk, from the reception signal sequence for the reception transducers Rk in the reception aperture Rx, as a reception signal corresponding to each reception transducer Rk based on the reflected ultrasonic wave from the observation point Pij.

The delay processor 1047 acquires, as inputs, the information indicating the position of the reception transducer Rk from the reception aperture setter 1043, the reception signal corresponding to the reception transducer Rk from the data storage 107, the information indicating the position of the observation point Pij acquired from the observation point setter 1042, and the delay amount to be applied to the reception signal sequence for each reception transducer Rk from the delay amount calculator 1046, in synchronization with the transmission of the ultrasonic beam. Then, the delay processor 1047 identifies the reception signal corresponding to the time obtained by subtracting the delay amount for each reception transducer Rk, from the reception signal sequence corresponding to each reception transducer Rk, as the reception signal based on the reflected wave from the observation point Pij, and outputs the identified reception signal to the adder 1049.

vii) Weight Calculator 1048

The weight calculator 1048 is a circuit for calculating a weight numerical sequence (reception apodization) for each reception transducer Rk such that the weight for the transducer located in the center in the sequence direction of the reception aperture Rx becomes maximum. The weight numerical sequence is a numerical sequence of a weighting coefficient to be applied to the reception signal corresponding to each transducer in the reception aperture Rx. The weight numerical sequence has symmetric distribution centered around the transmission focal point F. As the shape of the distribution of the weight numerical sequence, a Hamming window, a Hanning window, a rectangular window, or the like can be used, and the shape of the distribution is not particularly limited. The weight numerical sequence is set such that the weight for the transducer located in the center of the reception aperture Rx in the sequence direction becomes maximum, and a central axis of the weight distribution coincides with a reception aperture central axis Rxo. The weight calculator 1048 calculates the weight numerical sequence for each reception transducer Rk, using the information indicating the position of the reception transducer Rk output from the reception aperture setter 1043 as an input, and outputs the weight numerical sequence to the adder 1049.

viii) Adder 1049

The adder 1049 is a circuit for adding the reception signals identified corresponding to each reception transducer Rk output from the delay processor 1047 as inputs to generate a phased and added acoustic line signal for the observation point Pij. Alternatively, the adder 1049 may further amplify the reception signal identified corresponding to each reception transducer Rk by the weight for each reception transducer Rk, using the weight numerical sequence for each reception transducer Rk output from the weight calculator 1048 as an input, and add the reception signals to generate the acoustic line signal for the observation point Pij. The delay processor 1047 adjusts phases of the reception signals detected by each reception transducer Rk located in the reception aperture Rx and the adder 1049 adds the reception signals, whereby the reception signals received in each reception transducer Rk are superimposed on the basis of the reflected wave from the observation point Pij and the signal S/N ratio is increased, and the reception signal from the observation point Pij can be extracted.

The acoustic line signals can be generated for all the observation points Pij from the one-time transmission of the ultrasonic beam and the processing accompanying the transmission.

<Calculation of Reception Time>

Hereinafter, the processing of calculating the reception time in the reception time calculator 1045 will be described in more detail.

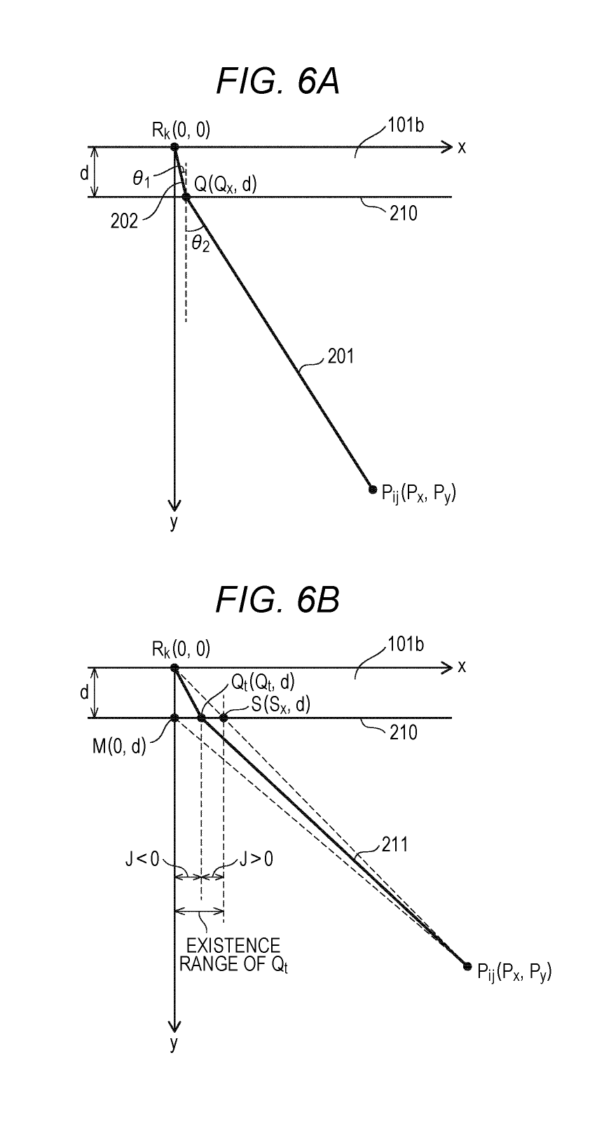

FIG. 6A is a schematic diagram illustrating a path in which the reflected wave from the observation point Pij reaches the reception transducer Rk. Here, an intersection point between a refractive surface 210, which is a boundary surface between the subject and the acoustic lens 101b, and the propagation path of the reflected ultrasonic wave is defined as a via point Q. At this time, the following expression (1) is established from the Snell's law, where an incident angle of an ultrasonic wave path 201 in the subject from the observation point Pij to the via point Q with respect to the refractive surface 210 is θ₂, and an emission angle of an ultrasonic wave path 202 in the acoustic lens 101b from the via point Q to the reception transducer Rk with respect to the refractive surface 210 is θ₁.

$$\frac{sin {θ}_{1}}{sin {θ}_{2}} = \frac{{v}_{1}}{{v}_{2}} = {n}_{21} \qquad [ Expression 1 ]$$

Here, v₁ is the sound velocity in the acoustic lens, v₂ is the sound velocity in the subject, and n₂₁ is the specific refractive index of the subject with respect to the acoustic lens.

From the above expression (1), the following expression (2) is naturally established.

sin²θ₁−n₂₁² sin²θ₂=0 [Expression 2]

Here, the thickness of the acoustic lens 101b is d, and Rk is the origin (0, 0), Pij is (Pₓ, P_{y}), and Q is (Qₓ, d) (where Pₓ>0 and P_{y}>0) with respect to the x axis (element sequence direction) and the y axis (depth direction). At this time, sin θ₁ and sin θ₂ satisfy the following expressions (3) and (4), respectively.

$$\begin{matrix}sin {θ}_{1} = \frac{{Q}_{x}}{\sqrt{{d}^{2} + {Q}_{x}^{2}}} & [ Expression 3 ] \\ sin {θ}_{2} = \frac{{P}_{x} - {Q}_{x}}{\sqrt{{( {P}_{y} - d )}^{2} + {( {P}_{x} - {Q}_{x} )}^{2}}} & [ Expression 4 ]\end{matrix}$$

When the above expressions (3) and (4) are substituted for the expression (2) and the denominator is reorganized, the following expression (5) is obtained.

Qₓ²{(P_{y}−d)²+(Pₓ−Qₓ)²}−n₂₁²(Pₓ−Qₓ)²(d²+Qₓ²)=0 [Expression 5]

Here, an evaluation function J (Qₓ) is defined as in the following expression (6).

J(Qₓ)=Qₓ²{(P_{y}−d)²+(Pₓ−Qₓ)²}−n₂₁²(Pₓ−Qₓ)²(d₂+Qₓ²) [Expression 6]

Since J (Qₓ) is the left side of the expression (5), J (Qₓ)=0 is obtained in the case where the via point Q (Qₓ, d) is a refraction point Qt (Qₜ, d) that satisfies the Snell's law. Meanwhile, J (Qₓ)>0 indicates that θ₁ is larger than a value determined by the Snell's law (θ₂ is smaller than the value determined by the Snell's law) from the expression (2), and thus indicates Qₓ>Qₜ. On the other hard, J (Qₓ)<0 indicates that θ₁ is smaller than the value determined by the Snell's law (θ₂ is larger than the value determined by the Snell's law) from the expression (2), and thus indicates Qₓ<Qₜ.

FIG. 6B is a diagram schematically illustrating the above relationship. J>0 is obtained at the via point Q having a large x coordinate with respect to the refraction point Qt (Qₜ, d) satisfying the Snell's law, and J<0 is obtained at the via point Q having a small x coordinate with respect to the refraction point Qt (Qₜ, d). Further, since the signs of the incident angle θ₂ and the emission angle θ, are the same from the expression (1), the refraction point Qt has a larger x coordinate than a maximum refraction point M (0, d) that is a point on the refractive surface 210, the maximum refraction point M being most adjacent to the reception transducer Rk. This is because θ₁=0 is obtained at the maximum refraction point M, and signs of the incident angle θ₂ and the emission angle θ₁ are different on the left side of (the x coordinate is smaller than) the maximum refraction point M. Further, the sound velocity in the acoustic lens 101b is smaller than the sound velocity in the subject (the specific refractive index n₂₁<1), and thus θ₁>θ₁ is obtained from the expression (1). Therefore, the refraction point Qt is on the left side with respect to (the x coordinate is smaller than) a non-refraction point S (Sₓ, d), which is an intersection point between a straight line connecting the observation point Pij and the reception transducer Rk and the refractive surface 210. Therefore, it can be said that the refraction point Qt exists on a line segment MS.

In view of the foregoing, a method of searching for the refraction point Qt will be described.

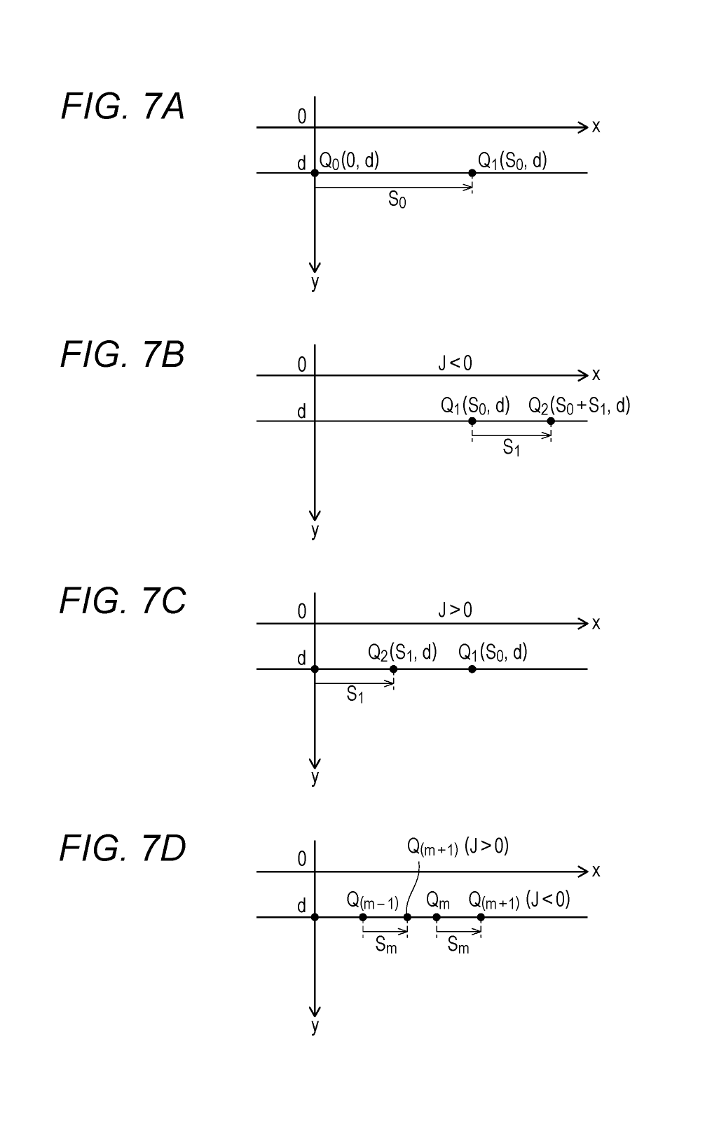

FIGS. 7A to 7D are schematic diagrams illustrating a method of detecting the refraction point Qt according to the first embodiment. First, as illustrated in FIG. 7A, the value of the evaluation function J is calculated setting the maximum refraction point M as a candidate via point Q₀, and whether the value is 0 or not is detected. Specifically, whether an absolute value |J| of J falls below a predetermined threshold value δ or not is detected. In the case where the absolute value |J| of J falls below the predetermined threshold value δ, the candidate via point Q₀ is detected as the refraction point Qt. On the other hand, in the case where the absolute value |J| of J is larger than the predetermined threshold value δ, the sign of J is evaluated. Since J≤0 is always satisfied at the maximum refraction point M, the sign of J is negative. Therefore, the x coordinate of the refraction point Qt is larger than the x coordinate of the candidate via point Q₀. Therefore, the next candidate via point Q₁ is set to Q₁ (S₀, t) that is separated by S₀ in a positive direction of the x axis. Next, similarly, the value of the evaluation function J of the candidate via point Q₁ is calculated and whether the value is 0 or not is detected. In the case where the absolute value |J| of J falls below the predetermined threshold value δ, the candidate via point Q₁ is detected as the refraction point Qt. On the other hand, in the case where the absolute value |J| of J is larger than the predetermined threshold value δ, the sign of J is evaluated. In the case where the sign of J is negative, the x coordinate of the refraction point Qt is larger than the x coordinate of the candidate via point Q₁. Therefore, as illustrated in FIG. 7B, the next candidate via point Q₂ is set to Q₂ (S₀+S₁, t) that is separated by S₁ in the positive direction of the x axis. Here, S₁=S₀/2. On the other hand, in the case where the sign of J is positive, the x coordinate of the refraction point Qt is smaller than the x coordinate of the candidate via point Q₁. Therefore, as illustrated in FIG. 7C, the next candidate via point Q₂ is set to Q₂ (S₁, t) that is separated from the candidate via point Q₀ by Sₜ in the positive direction of the x axis. Hereinafter, similar processing is repeated. That is, as illustrated in FIG. 7D, the value of the evaluation function J is calculated for the candidate via point Qₘ (m is an integer of 1 or more), and in the case where J=0 can be regarded, the candidate via point Qₘ is detected as the refraction point Qt. Meanwhile, in the case of J<0, the candidate via point Q is set to Qₘ₊₁ that is separated from the candidate via point Qₘ by Sₘ (Sₘ=Sₘ₋₁/2) in the positive direction of the x axis, and in the case of J>0, the candidate via point Qₘ is set to Qₘ₊₁ that is separated from the candidate via point Qₘ₋₁ by Sₘ in the positive direction of the x axis. By repeating this processing, the refraction point Qt can be specified without making the number of trials m excessive.

Note that it is favorable that S₀≥D/2, and more favorable that S₀=D/2, where the length of the line segment MS is D. Further, it is not limited to Sₘ=Sₘ₋₁/2 (m is an integer of 1 or more), and it may be Sₘ₋₁>Sₘ>Sₘ₋₁/2.

<Operation>

An operation of the ultrasonic diagnostic device 100 having the above configuration will be described.

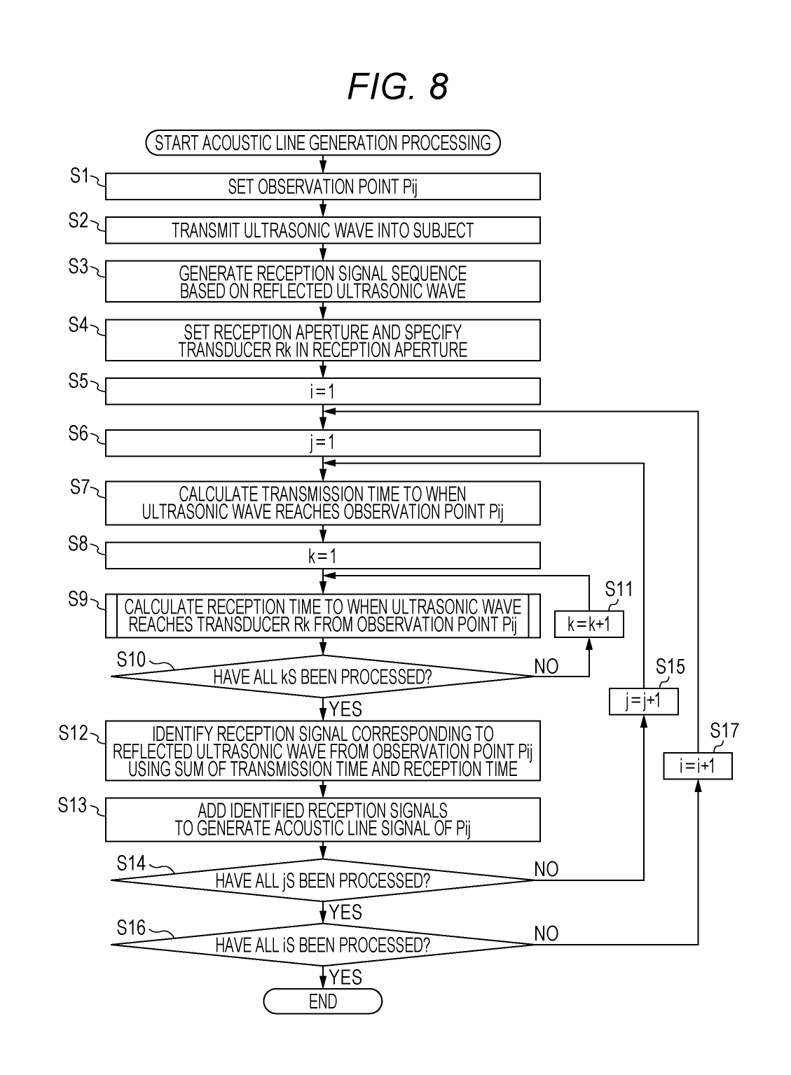

FIG. 8 is a flowchart illustrating a beamforming processing operation of the reception beamformer 104.

First, in step S1, the observation point setter 1042 acquires the information indicating the position of the transmission aperture Tx from the transmitter 1031, and sets the plurality of observation points Pij.

Next, in step S2, the transmitter 1031 supplies the transmission signal for causing each transducer included in the transmission aperture Tx, of the plurality of transducers 101a present in the probe 101, to transmit the ultrasonic beam, and causes each transducer to transmit the ultrasonic beam into the subject.

Next, in step S3, the receiver 1040 generates the reception signal on the basis of the electrical signal obtained from the reception of the ultrasonic reflected wave in the probe 101 and outputs the reception signal to the data storage 107, and stores the reception signal in the data storage 107.

Next, in step S4, the reception aperture setter 1043 sets the reception aperture Rx. Here, the reception aperture Rx is selected such that the sequence center of the transmission aperture Tx coincides with the sequence center of the reception aperture Rx.

Next, the acoustic line signal is generated for the observation point Pij. First, variables i and j are initialized in steps S5 and S6.

Next, in step S7, the transmission time calculator 1044 calculates, for the observation point Pij, the time to when the transmitted ultrasonic wave reaches the observation point Pij in the subject. The transmission time is calculated by dividing a path length from the transmission aperture Tx to the observation point Pij by the sound velocity of the ultrasonic wave. Here, it is assumed that the path length is a linear distance from the transmission aperture Tx to the observation point Pij. Note that the linear distance from the transmission aperture Tx to the observation point Pij is an example of the path length and the path length is not limited thereto, and a path suitable for the transmission beamforming method and the reception beamforming method may be selected.

Next, in step S8, a coordinate k indicating the position of the reception transducer Rk in the reception aperture Rx is initialized to the minimum value in the reception aperture Rx. In step S9, the reception time to when the ultrasonic wave is reflected at the observation point Pij and reaches the reception transducer Rk of the reception aperture Rx is calculated.

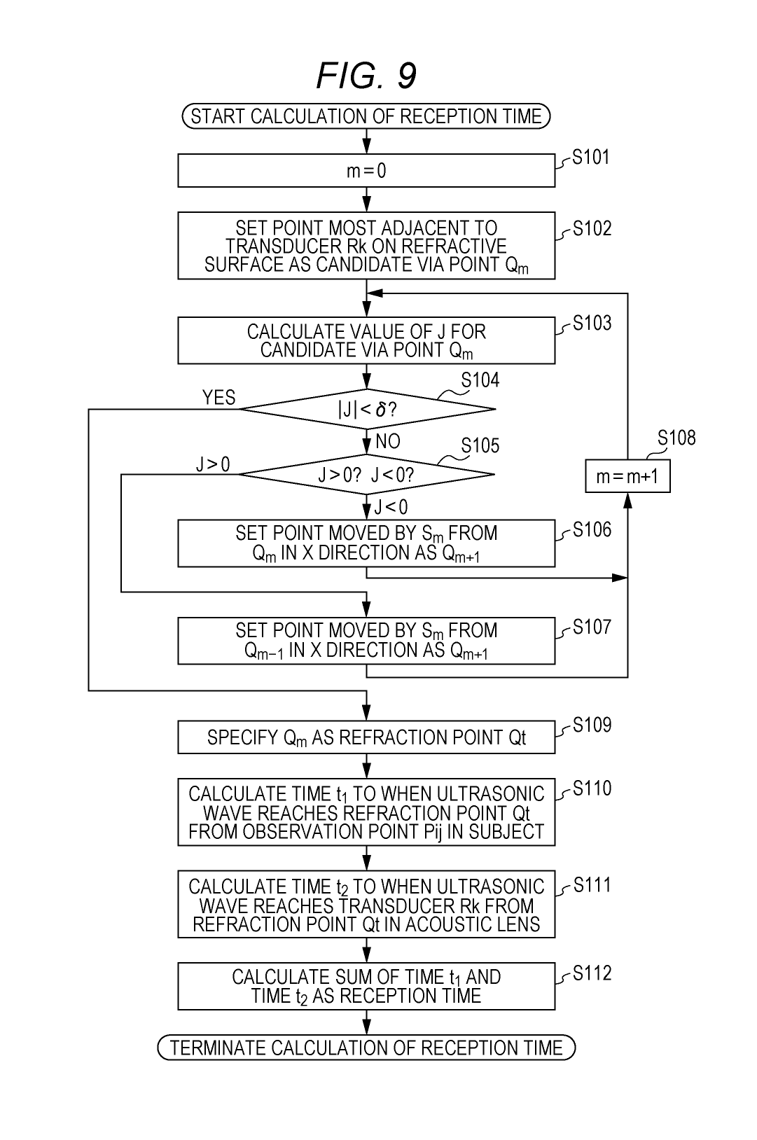

Here, the operation of calculating the reception time in step S9 will be described in more detail. FIG. 9 is a flowchart illustrating an operation to calculate the reception time in the reception time calculator 1045.

First, in step S101, a variable m is initialized to the minimum value 0. Next, in step S102, a point most adjacent to the transducer Rk on the refractive surface 210 is set as the candidate via point Qₘ. As a result, the maximum refraction point M most adjacent to the transducer Rk on the refractive surface 210 is set as the candidate via point Q₀.

Next, in step S103, the value of the evaluation function J is calculated for the candidate via point Qₘ. With the calculation, the evaluation function J (M) corresponding to the maximum refraction point M is calculated.

Next, in step S104, whether the value of the evaluation function J can be regarded as 0 or not is determined. Specifically, whether the absolute value |J| of the evaluation function J falls below the predetermined threshold value δ or not is determined. In the case where the absolute value |J| of the evaluation function J falls below the threshold value δ, the processing proceeds to step S109. On the other hand, in the case where the absolute value |J| of the evaluation function J is the threshold value δ or more, the sign of the evaluation function J is determined in step S105. In the case where the sign of the evaluation function J is negative, the x coordinate of the refraction point Qt is larger than the x coordinate of the candidate via point Qₘ. Therefore, in step S106, a point moved from the candidate via point Qₘ by Sₘ in the x direction is set to the next candidate via point Qₘ₊₁, and in step S108, m is incremented and step S103 is retried. As for the candidate via point Q₀, the evaluation function J always satisfies J≤0. Therefore, in the case where the processing does not proceed to step S109, the processing always proceeds to step S106.

Next, in retried step S103, the value of the evaluation function J is calculated for the candidate via point Qₘ. With the calculation, the evaluation function J corresponding to the candidate via point Q₁ is calculated. Then, in step S104, in the case where the absolute value |J| of the evaluation function J falls below the threshold value δ, the processing proceeds to step S109. On the other hand, in the case where the absolute value |J| of the evaluation function is the threshold value δ or more, the sign of the evaluation function J is determined in step S105. In the case where the sign of the evaluation function J is negative, the x coordinate of the refraction point Qt is larger than the x coordinate of the candidate via point Qₘ. Therefore, in step S106, a point moved from the candidate via point Qₘ by Sₘ in the x direction is set to the next candidate via point Qₘ₊₁, and in step S108, in is incremented and step S103 is retried. On the other hand, in the case where the sign of the evaluation function J is positive, the x coordinate of the refraction point Qt is smaller than the x coordinate of the candidate via point Qₘ. Therefore, in step S107, a point moved from the previous candidate via point Qₘ₋₁ by Sₘ in the x direction is set to the next candidate via point Qₘ₊₁, and in step S108, m is incremented and step S103 is retried. With the processing, the candidate via point Qₘ at which absolute value |J| of the evaluation function J falls below the threshold value δ is specified.

In step S109, the candidate via point Q at which the absolute value |J| of the evaluation function J falls below the threshold value δ is specified as the refraction point Qt. Next, in step S110, a time t₁ to when the ultrasonic wave reaches the refraction point Qt from the observation point Pij in the subject is calculated. The time t₁ can be calculated by dividing the geometrical linear distance from the observation point Pij to the refraction point Qt by the sound velocity in the subject. Further, in step S111, a time t₂ to when the ultrasonic wave reaches the reception transducer Rk from the refraction point Qt in the acoustic lens is calculated. The time t₂ can be calculated by dividing the geometrical linear distance from the refraction point Qt to the reception transducer Rk by the sound velocity in the acoustic lens. Then, in step S112, the sum of the time t₁ and the time t₂ is calculated as the reception time.

Referring back to FIG. 8, description will be continued. In step S10, whether the reception time has been calculated or not for all the reception transducers Rk present in the reception aperture Rx is determined. In the case of non-completion, k is incremented in step S11 and step S9 is further performed, and in the case of completion, the processing proceeds to step S11. With the processing, the reception time has been calculated for all the reception transducers Rk present in the reception aperture Rx.

Next, in step S12, the reception signal based on the reflected ultrasonic wave from the observation point Pij is identified using the sum of the transmission time and the reception time. First, the delay amount calculator 1046 calculates the total propagation time of each reception transducer Rk, using the transmission time calculated in step S7 and the reception time of each reception transducer Rk calculated in steps S8 to S11, and calculates the delay amount for each reception transducer Rk according to the difference in the total propagation time for each reception transducer Rk in the reception aperture Rx. Next, the delay processor 1047 identifies the reception signal corresponding to the time obtained by subtracting the delay amount for each reception transducer Rk, from the reception signal sequence corresponding to the reception transducer Rk in the reception aperture Rx, as the reception signal based on the reflected wave from the observation point Pij.

Next, in step S13, the identified reception signals are added to generate the acoustic line signal of Pij. First, the weight calculator 1048 calculates the weight numerical sequence for each reception transducer Rk such that the weight for the transducer located in the center in the sequence direction of the reception aperture Rx becomes maximum. The adder 1049 multiplies the reception signal identified corresponding to each reception transducer Rk by the weight for each reception transducer Rk and adds the reception signals to generate the acoustic line signal for the observation point Pij. The generated acoustic line signal of the observation point Pij is output to the data storage 107 and stored.

Next, by incrementing the coordinate ij and repeating steps S7 to S13, the acoustic line signal is generated for all the observation points Pij. Whether generation of the acoustic line signal has been completed for all the observation points Pij or not is determined (steps S14 and S16). In the case of non-completion, the coordinate ij is incremented (steps S15 and S17), and the acoustic line signal for the observation point Pij is generated. When the acoustic line signal has been generated for all the observation points Pij, generation of the acoustic line signal group corresponding to the transmission of the ultrasonic beam in step S2 is completed.

<Conclusion>

As described above, according to the ultrasonic diagnostic device 100 of the present embodiment, the acoustic line signal for the observation point Pij is generated on the basis of the highly precise reception time considering the influence of the acoustic lens. As a result, the precision of reception beamforming can be improved, and the spatial resolution and the signal S/N ratio can be improved, for all the observation points Pij.

Further, in the ultrasonic diagnostic device 100, in searching for the refraction point Qt, a dichotomous method (or a similar method) using the evaluation function J and using the maximum refraction point M, which is the point most adjacent to the reception transducer on the refractive surface, as a starting point, is used. As a result, the number of search trials of the refraction point Qt can be decreased. Therefore, the operation amount required for calculating the reception time is not large. Therefore, the precision of the reception beamforming can be improved without substantially increasing the operation amount of the phasing addition, as compared with the conventional phasing addition method.

Further, in the ultrasonic diagnostic device 100, highly precise reception time calculation, considering the influence of the acoustic lens, is performed for all the combinations of the observation points Pij and the reception transducers Rk. Therefore, the precision of the reception beamforming can be improved for any of the observation points Pij without holding a result of preliminary calculation in a large capacity memory. Therefore, the precision of the reception beamforming can be improved for all the observation points Pij without large-capacity correction value data, as compared with the method of holding correction values calculated in advance in a memory.

### First Modification

In the ultrasonic diagnostic device 100 according to the first embodiment, the probe 101 has been the linear probe in which the plurality of transducers 101a is linearly arranged. However, the form of an ultrasonic probe is not limited to the above-described arrangement, and another form may be employed.

A first modification is different from the first embodiment in that an ultrasonic probe is a convex probe in which a plurality of transducers is concentrically arranged. Configurations other than the ultrasonic probe are the same as the elements illustrated in the first embodiment, and description of the same part is omitted.

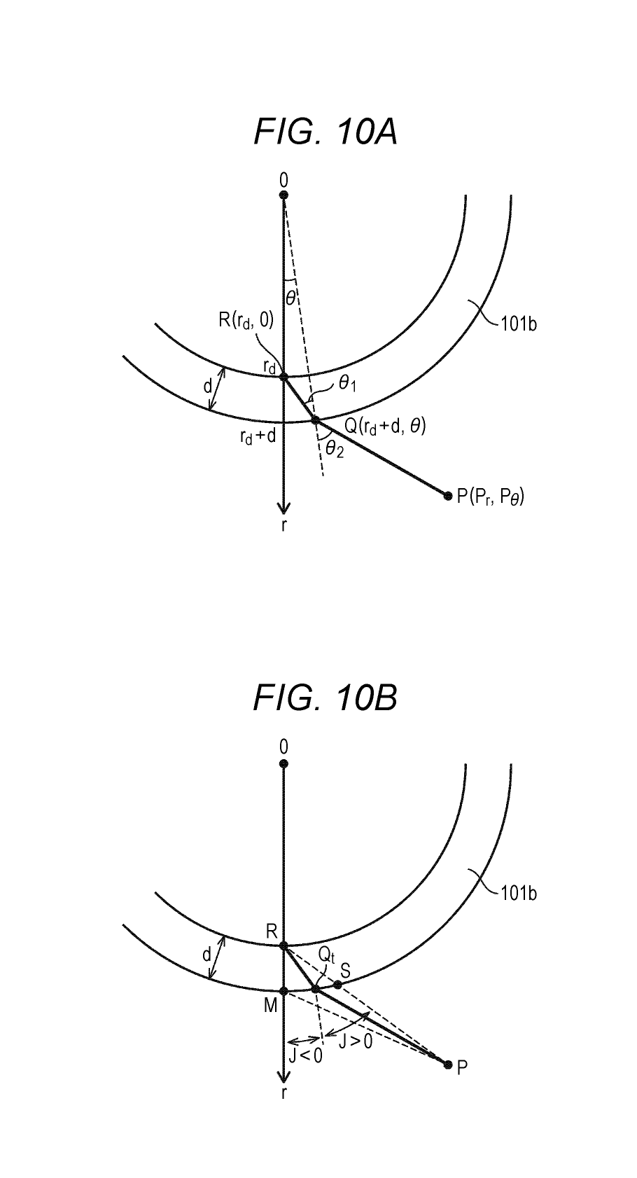

FIG. 10A is a schematic diagram illustrating a path in which a reflected wave from an observation point Pij reaches a reception transducer Rk. Here, it is assumed that a transducer is present on an arc with a radius r_{d} around a point O, and a refractive surface (an outer periphery of an acoustic lens) is an arc with a radius r_{d}+d around the point O. That is, the thickness of the acoustic lens is d. At this time, the above expressions (1) and (2) are established from the Snell's law, where an incident angle of an ultrasonic wave path in a subject from the observation point Pij to a via point Q with respect to the refractive surface is θ₂, and an emission angle of an ultrasonic wave path in the acoustic lens from the via point Q to the reception transducer Rk with respect to the refractive surface is θ₁.

Here, positions of the reception transducer Rk, the via point Q, and the observation point Pij are illustrated by circular polar coordinates rθ with respect to the point O. As for θ, the position of the reception transducer Rk is set to θ=0, and the Pij side takes a positive value. The coordinates of the reception transducer Rk are (r_{d}, 0), the coordinates of the via point Q are (r_{d}+d, θ), and the coordinates of the observation point Pij are (Pᵣ, P_{θ}) in the circular polar coordinates tr. At this time, the coordinates of the reception transducer Rk, the via point Q, and the observation point Pij are converted into xy coordinates, and the coordinates become Rk (0, r_{d}), Q ((r_{d}+d)sin θ, (r_{d}+d)cos θ), and Pij (Pₜ sin P_{θ}, Pᵣ cos P_{θ}), respectively. Therefore, sin θ₁ and sin θ₂ satisfy the following expressions (7) and (8) respectively.

$$\begin{matrix}sin {θ}_{1} = \frac{{r}_{d} sin θ}{\sqrt{\begin{matrix}{( {r}_{d} + d )}^{2} {sin}^{2} θ + \\ {\{ ( {r}_{d} + d ) cos θ - {r}_{d} \}}^{2}\end{matrix}}} & [ Expression 7 ] \\ sin {θ}_{2} = \frac{{P}_{r} sin ( {P}_{θ} - θ )}{\sqrt{\begin{matrix}{\{ ( {r}_{d} + d ) sin θ - {P}_{r} sin {P}_{θ} \}}^{2} + \\ {\{ {P}_{r} cos {P}_{θ} - ( {r}_{d} + d ) cos θ \}}^{2}\end{matrix}}} & [ Expression 8 ]\end{matrix}$$

Here, when the denominators of the above-described expressions (7) and (8) are reorganized, the following expressions (9) and (10) are obtained.

$$\begin{matrix}sin {θ}_{1} = \frac{{r}_{d} sin θ}{\sqrt{{( {r}_{d} + d )}^{2} + {r}_{d}^{2} - 2 {r}_{d} ( {r}_{d} + d ) cos θ}} & [ Expression 9 ] \\ sin {θ}_{2} = \frac{{P}_{r} sin ( {P}_{θ} - θ )}{\sqrt{\begin{matrix}{P}_{r}^{2} + {( {r}_{d} + d )}^{2} - \\ 2 {P}_{r} ( {r}_{d} + d ) cos ( {P}_{θ} - θ )\end{matrix}}} & [ Expression 10 ]\end{matrix}$$

Therefore, when an evaluation function J is defined as in the first embodiment, the evaluation function can be defined by the following expression (11).

J(θ)=r_{d}² sin²{Pᵣ²+(r_{d}+d)²−2Pᵣ(r_{d}+d)cos(P_{θ}−θ)}−n₂₁²Pₜ² sin²(P_{θ}−θ){(r_{d}+d)²+r_{d}²−2r_{d}(r_{d}+d)cos θ} [Expression 11]

A reception time calculator searches for a refraction point Qt, by a dichotomous method (or a similar method) using the evaluation function J and using the maximum refraction point M, which is the point most adjacent to the reception transducer on the refractive surface, as a starting point, similarly to the first embodiment. At this time, in the case where the value of the evaluation function J cannot be regarded as 0 for a candidate via point Qₘ (r_{d}+d, θₘ), the candidate via point Qₘ₊₁ is determined as follows. That is, as illustrated in FIG. 10B, in the case of J<0, θₘ<θt is satisfied for a refraction point Qt (r_{d}+d, θₜ). Therefore, a candidate via point Qₘ₊₁(r_{d}+d, θₘ+Sₘ), which is moved by Sₘ in a θ direction, is provided. On the other hand, in the case of J>0. θₘ>θt is satisfied. Therefore, a candidate via point Qₘ₊₁(r_{d}+d, θₘ₋₁+Sₘ) moved from the candidate via point Qₘ₋₁ by Sₜ in the θ direction is provided. Other processes are similar to those in the first embodiment and are thus omitted.

<Conclusion>

As described above, according to the ultrasonic diagnostic device of the first modification, similar effects to the first embodiment can be obtained in the case where the plurality of transducers is concentrically arranged and the convex probe having an acoustic lens is used.

### Second Embodiment

In the first embodiment, the case of calculating the reception time to when the reflected ultrasonic wave reaches the reception transducer Rk from the observation point Pij by specifying the position of the refraction point Qt that is the point when the propagation path of the reflected ultrasonic wave passes through the interface between the subject and the acoustic lens has been described. However, if there is a method that can directly calculate the reception time, it is not necessary to specify the refraction point Qt.

A second embodiment is different from the first embodiment in that a reception time calculator directly calculates a reception time. Configurations other than the reception time calculator are the same as the elements illustrated in the first embodiment, and description of the same part is omitted.

<Calculation Principle>

A reception time t to when a reflected ultrasonic wave reaches a reception transducer Rk from an observation point Pij, going through a via point Q, illustrated in FIG. 6A, can be expressed by the next expression (12), using coordinates (Qₓ, d) of the via point Q, a sound velocity v₁ in an acoustic lens, and a sound velocity v₂ in a subject.

$$t ( {Q}_{x} ) = \frac{\sqrt{{Q}_{x}^{2} + {d}^{2}}}{{v}_{1}} + \frac{\sqrt{{( {P}_{x} - {Q}_{x} )}^{2} + ( {P}_{y} - d )}}{{v}_{2}} \qquad [ Expression 12 ]$$

Here, the following expression (13) is obtained when the reception time t is differentiated by an x coordinate Qₓ of the via point Q.

$$\frac{dt ( {Q}_{x} )}{{dQ}_{x}} = \frac{{Q}_{x}}{{v}_{1} \sqrt{{Q}_{x}^{2} + {d}^{2}}} + \frac{{Q}_{x} - {P}_{x}}{{v}_{2} \sqrt{{( {P}_{x} - {Q}_{x} )}^{2} + {( {P}_{y} - d )}^{2}}} \qquad [ Expression 13 ]$$

When the expression (13) above is reorganized using the expressions (2), (3), and (4), the following expression (14) is obtained.

$$\frac{dt ( {Q}_{x} )}{{dQ}_{x}} = \frac{1}{{v}_{1}} ( sin {θ}_{1} - {n}_{21} sin {θ}_{2} ) \qquad [ Expression 14 ]$$

Here, dt/dQₓ=0 is known when Qₓ=Qt with respect to a refraction point Qt (Qt, d) from the expression (1) which is the Snell's law. Further, dt/dQₓ<0 is satisfied when Qₓ<Qt, and dt/dQₓ>0 is satisfied when Qₓ>Qt.

Therefore, the reception time t (Qₓ) takes a minimum value when Qₓ=Qt. In other words, in the case where Q (Qₓ, d) is set on a line segment MS illustrated in FIG. 6B, Q (Qₓ, d) at which the reception tune t (Qₓ) becomes minimum is the refraction point Qt (Qt, d).

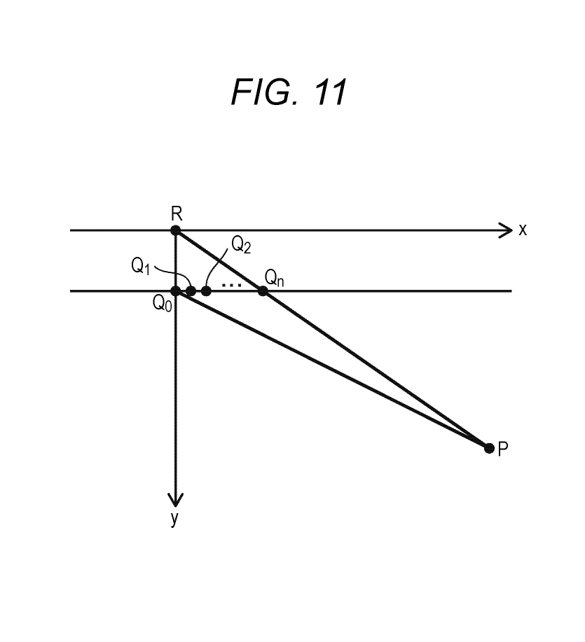

From the above viewpoint, as illustrated in FIG. 11, a plurality of candidate via points Qₘ (Qₘ, d) including the point M and the point S on the line segment MS is provided and the reception time t (Qₘ) is calculated for each of the candidate via points Qₘ, and the minimum value is used as is as the reception time.

<Operation>

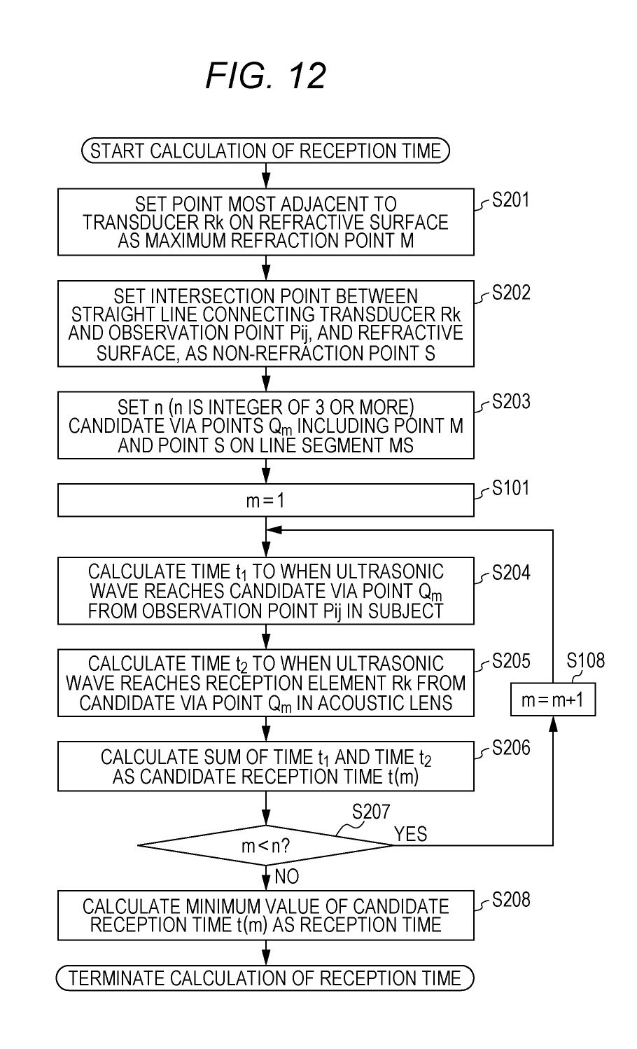

A method of calculating the reception time in the reception time calculator according to the second embodiment will be described. FIG. 12 is a flowchart illustrating a method of calculating the reception time according to the second embodiment.

First, in step S201, the reception time calculator specifies a point most adjacent to the reception transducer Rk on the refractive surface, as a maximum refraction point M.

Next, in step S202, the reception time calculator specifies an intersection point between a straight line connecting the reception transducer Rk and the observation point Pij and the refractive surface, as a non-refraction point S.

Next, in step S203, the reception time calculator provides n (n is an integer of 3 or more) candidate via points Qₘ (Qₘ, d) including the point M and the point S on the line segment MS. For example, as illustrated in FIG. 11, the candidate via point Qₘ has a candidate via point Q₁ as the maximum refraction point M, and a candidate via point Qₙ as the non-refraction point S. For Q₂ to Qₙ₋₁, Q₁ to Qₙ can be set to be equally spaced, for example.

Next, in step S101, a variable m is initialized, and in step S204, a time t₁ to when the ultrasonic wave reaches the candidate via point Qₘ from the observation point Pij in the subject is calculated. The time t₁ can be calculated by dividing a geometrical linear distance from the observation point Pij to the candidate via point Qₘ by a sound velocity in the subject. Further, in step S205, a time t₂ to when the ultrasonic wave reaches the reception transducer Rk from the candidate via point Qₘ in the acoustic lens is calculated. The time t₂ can be calculated by dividing a geometrical linear distance from the candidate via point Qₘ to the reception transducer Rk by a sound velocity in the acoustic lens. Then, in step S206, the sum of the time t₁ and the time t₂ is calculated as a candidate reception time t (m).

In step S207, whether the candidate reception time t (m) for all the candidate via points Qₘ has been calculated or not is determined. In the case of non-completion, m is incremented in step S108 and steps S204 to S206 are further performed, and in the case of completion, the processing proceeds to step S208. With the processing, the candidate reception time t (m) has been calculated for all the candidate via points Qₘ. Here, calculation of the candidate reception time t (m) for each candidate via point Qₘ has been sequentially performed. However, since calculation processing of the candidate reception time t (m) is independent of every m, the calculation processing may be performed in parallel for each m or for each set of m. By doing so, the calculation time can be shortened.

Next, in step S208, the smallest value along the candidate reception tines t (m) is output as the reception time, and the processing is terminated.

<Conclusion>

As described above, the ultrasonic diagnostic device according to the second embodiment has the following effects in place of the effects except the part regarding specification of the refraction point Qt, of the effects described in the first embodiment. That is, in the ultrasonic diagnostic device according to the second embodiment, the reception time based on the plurality of candidate reflected ultrasonic wave paths is calculated, and the minimum value thereof is adopted. Thereby, the reception time can be directly calculated without specifying the refraction point Qt. Therefore, the calculation processing of the reception time can be simplified. Furthermore, the calculation processing of the reception time can be performed by parallel processing, and when such a method is adopted, the precision of reception beamforming can be improved, and the spatial resolution and the signal S/N ratio can be improved, without increasing the time required for calculating the reception time.

### Second Modification

In the ultrasonic diagnostic device 100 according to the second embodiment, the probe 101 has been the linear probe in which the plurality of transducers 101a is linearly arranged. However, the form of an ultrasonic probe is not limited to the above-described arrangement, and another form may be employed.

A second modification is different from the second embodiment in that an ultrasonic probe is a convex probe in which a plurality of transducers is concentrically arranged. Configurations other than the ultrasonic probe are the same as the elements illustrated in the second embodiment, and description of the same part is omitted.

<Calculation Principle>

A reception time t to when a reflected ultrasonic wave reaches a reception transducer Rk from an observation point Pij, going through a via point Q, illustrated in FIG. 10A, can be expressed by the next expression (15), using coordinates (r_{d}+d, θ) of the via point Q, a sound velocity v₁ in an acoustic lens, and a sound velocity v₂ in a subject.

$$t ( θ ) = \frac{\sqrt{{( {r}_{d} + d )}^{2} {sin}^{2} θ + {\{ ( {r}_{d} + d ) cos θ - {r}_{d} \}}^{2}}}{{v}_{1}} + \frac{\sqrt{\begin{matrix}{\{ ( {r}_{d} + d ) sin θ - {P}_{r} sin {P}_{θ} \}}^{2} + \\ {\{ {P}_{r} cos {P}_{θ} - ( {r}_{d} + d ) cos θ \}}^{2}\end{matrix}}}{{v}_{2}} \qquad [ Expression 15 ]$$

Here, the following expression (16) is obtained when the reception time t is differentiated by a θ coordinate θ of the via point Q.

$$\frac{dt ( θ )}{d θ} = \frac{{r}_{d} ( {r}_{d} + d ) sin θ}{{v}_{1} \sqrt{{( {r}_{d} + d )}^{2} {sin}^{2} θ + {\{ ( {r}_{d} + d ) cos θ - {r}_{d} \}}^{2}}} - \frac{{P}_{r} ( {r}_{d} + d ) sin ( {P}_{θ} - θ )}{{v}_{2} \sqrt{\begin{matrix}{\{ ( {r}_{d} + d ) sin θ - {P}_{r} sin {P}_{θ} \}}^{2} + \\ {\{ {P}_{r} cos {P}_{θ} - ( {r}_{d} + d ) cos θ \}}^{2}\end{matrix}}} \qquad [ Expression 16 ]$$

When the expression (16) above is reorganized using the expressions (2), (7), and (8), the following expression (17) is obtained.

$$\frac{dt ( θ )}{d θ} = \frac{{r}_{d} + d}{{v}_{1}} ( sin {θ}_{1} - {n}_{21} sin {θ}_{2} ) \qquad [ Expression 17 ]$$

Here, dt/dθ=0 is known when θ=θₜ with respect to a refraction point Qt (r_{d}+d, θₜ) from the expression (1) which is the Snell's law. Further, dt/dθ<0 is satisfied when θ<0, and dt/dθ>0 is satisfied when θ>θₜ.

Therefore, the reception time t (θ) takes a minimum value when θ=θₜ.

From the above viewpoint, similarly to the second embodiment, a plurality of candidate via points Qₘ(r_{d}+d, θₘ) including a point M and a point S on an arc MS is provided and the reception time t (Qₘ) is calculated for each of the candidate via points Qₘ, and the minimum value is used as is as the reception time.

<Conclusion>

As described above, according to the ultrasonic diagnostic device of the second modification, similar effects to the second embodiment can be obtained in the case where the plurality of transducers is concentrically arranged and the convex probe having an acoustic lens is used.

### Third Embodiment

In the first and second embodiments, the case of calculating the value by the path going through the refraction point Qt, for the reception time to when the reflected ultrasonic wave reaches the reception transducer Rk from the observation point Pij, has been described.

In contrast, a third embodiment is different from the first and second embodiments in calculating a reception time simply. Configurations other than the reception time calculator are the same as the elements illustrated in the first and second embodiments, and description of the same part is omitted.

<Calculation Method>

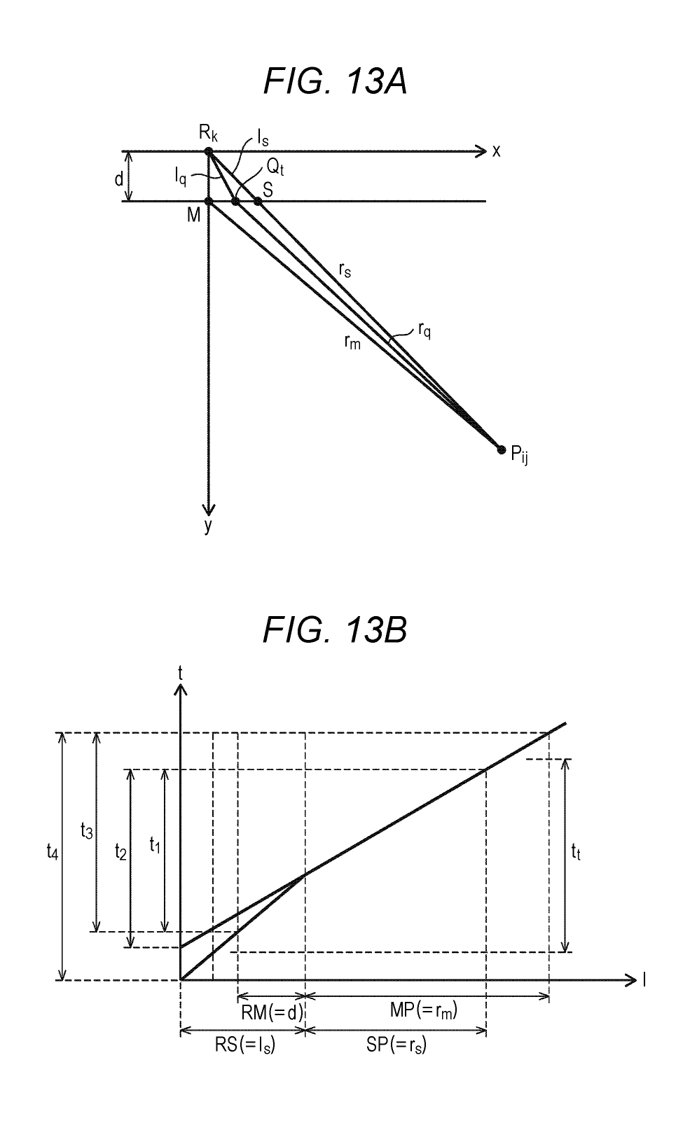

FIG. 13A is a schematic diagram illustrating a path in which a reflected wave from an observation point Pij reaches a reception transducer Rk. Here, a reception time tₜ can be expressed by the following expression (18), where a distance between the reception transducer Rk and the refraction point Qt is 1_{q} and a distance between the refraction point Qt and the observation point Pij is r_{q}.

$${t}_{t} = \frac{{r}_{q}}{{v}_{2}} + \frac{{l}_{q}}{{v}_{1}} \qquad [ Expression 18 ]$$

Here, a reception time t₁ of if a reflected wave reaches a non-refraction point S from the observation point Pij in a subject and to when the reflected wave reaches a reception transducer from a maximum refraction point M in an acoustic lens can be expressed by the following expression (19), where the thickness of the acoustic lens is d, and the distance between the non-refraction point S and the observation point Pij is rₛ.

$${t}_{1} = \frac{{r}_{s}}{{v}_{2}} + \frac{d}{{v}_{1}} \qquad [ Expression 19 ]$$

Since the assumed reception time t₁ corresponds to a case where the ultrasonic wave passes through the shortest route in both the acoustic lens and the subject and the route is discontinuous, the time is too short and cannot happen in reality. That is, t₁<tₜ.

Further, a reception time t₂ in the case of not considering the acoustic lens can be expressed by the following expression (20), where the distance between the reception transducer Rk and the non-refraction point S is Iₛ.

$${t}_{2} = \frac{{r}_{s}}{{v}_{2}} + \frac{{l}_{s}}{{v}_{2}} \qquad [ Expression 20 ]$$

Since the assumed reception time t₂ here is a value on the assumption that a sound velocity in the acoustic lens is equivalent to a sound velocity in the subject, the reception time t₂ becomes shorter than the actual reception time. That is, t₂<tₜ.

Meanwhile, a reception time t₃ of if the reflected wave reaches the maximum refraction point M from the observation point Pij in the subject and to when the reflected wave reaches the reception transducer from the maximum refraction point M in tire acoustic lens can be expressed by the following expression (21), where the distance between the maximum refraction point M and the observation point Pij is rₘ.

$${t}_{3} = \frac{{r}_{m}}{{v}_{2}} + \frac{d}{{v}_{1}} \qquad [ Expression 21 ]$$

Since the assumed reception time t₃ here is longer than the route via the refraction point Qt where the required time is the shortest, and thus tₜ<t₃.

Further, a reception time t₄ of if the reflected wave reaches the maximum refraction point M from the observation point Pij in the subject and to when the reflected wave reaches the reception transducer from the non-refraction point S in the acoustic lens can be expressed by the following expression (22).

$${t}_{4} = \frac{{r}_{m}}{{v}_{2}} + \frac{{l}_{s}}{{v}_{1}} \qquad [ Expression 22 ]$$

The assumed reception time t₄ here corresponds to a case where the ultrasonic wave passes through a route longer than the route going through the refraction point Qt in both the acoustic lens and the subject, and is clearly longer than the route going through the refraction point Qt where the required time is the shortest. That is, tₜ<t₄.

As also illustrated in the graph in FIG. 13B, the above-described t₁ and t₂ are shorter than tₜ to be calculated, and the above-described t₃ and t₄ are longer than tₜ to be calculated. In other words, it can be said that tₜ is a value between a representative value of t₁ and t₂ and a representative value of t₃ and t₄. Therefore, tₜ can be approximated by arithmetic mean or weighted average of at least one of t₁ and t₂ and at least one of t₃ and t₄.

Therefore, for example, tₜ is calculated by the weighted average as illustrated in the following expression (23).

tₜ=α{βt₁+(1−β)t₂}+(1−α){γt₃+(1−γ)t₄} [Expression 23]

Here, α is a weighting coefficient, and 0<α<1. Although β and γ are also weighting coefficients, 0≤β≤1 and 0≤γ≤1 are satisfied because at least one of t₁ and t₂ and at least one of t₃ and t₄ are used for calculating tₜ as described above. The values of α, β, and γ may be constant with respect to every observation point Pij and the reception transducer Rk, or may be varied depending on a relative positional relationship between the observation point Pij and the reception transducer Rk.

Alternatively, for example, weighted average may be performed in the acoustic lens and in the subject, respectively, in a similar manner.

First, focusing on the inside of the acoustic lens. A time l_{q}/v₁ from the refraction point Qt to the reception transducer Rk, which is a true arrival time in the acoustic lens, is longer than a time d/v₁ from the maximum refraction point M to the reception transducer Rk, and a time lₛ/v₂ from the non-refraction point S to the reception transducer Rk at a velocity in the subject. Meanwhile, the time l_{q}/v₁, is shorter than the time lₛ/v₁ from the non-refraction point S to the reception transducer Rk. Therefore, the time l_{q}/v₁ can be approximated by arithmetic mean or weighted average of at least one of the time d/v₁ and the time lₛ/v₂, and the time lₛ/v₁.

Similarly, focusing on the inside of the subject. A time rₛ/v₂ from the observation point Pij to the refraction point Qt, which is a true arrival time in the subject, is longer than a time rₛ/v₂ from the observation point Pij to the non-refraction point S and is shorter than a time rₘ/v₂ from the observation point Pij to the maximum refraction point M. Therefore, the time r_{q}/v₂ can be approximated by arithmetic mean or weighted average of the time rₛ/v₂ and the time rₘ/v₂.

Therefore, for example, tₜ is calculated by the weighted average as expressed in the following expression (24).

$${t}_{t} = \frac{α {r}_{m} + ( 1 - α ) {r}_{s}}{{v}_{2}} + \frac{β \{ γ d + ( 1 - γ ) {n}_{21} {l}_{s} \} + ( 1 - β ) {l}_{s}}{{v}_{1}} \qquad [ Expression 24 ]$$

Here, α and β are weighting coefficients, and 0<α<1 and 0<β<0 are satisfied. Although γ is also a weighting coefficient, 0≤γ≤1 is satisfied because at least one of the time d/v₁ and the time lₛ/v₂ is used for calculating tₜ as described above. The values of α, β, and γ may be constant with respect to every observation point Pij and the reception transducer Rk, or may be varied depending on a relative positional relationship between the observation point Pij and the reception transducer Rk. For example, tₜ may be calculated by the weighted average like the following expression (25), where γ=0.

$${t}_{t} = \frac{α {r}_{m} + ( 1 - α ) {r}_{s}}{{v}_{2}} + \frac{β d + ( 1 - β ) {l}_{s}}{{v}_{1}} \qquad [ Expression 25 ]$$

Note that, in the above expressions (24) and (25), 0<α<1 is satisfied. However, in the case where the depth of the observation point Pij is sufficiently larger than the thickness d of the acoustic lens, the difference between the distance rₛ from the observation point Pij to the non-refraction point S and the distance rₘ from the observation point Pij to the maximum refraction point M is small enough to be negligible, and rₘ−r_{q}>>d is satisfied. Therefore, for such observation point Pij, the influence of a on the value of tₜ is extremely small, and thus α=0 or α=1 may be used.

Although, here, the case where the probe is a linear probe has been described, similar processing can be performed even if the probe is a convex probe.

<Conclusion>

As described above, the ultrasonic diagnostic device according to the third embodiment has the following effects in place of the effects except the part regarding specification of the refraction point Qt. of the effects described in the first embodiment. That is, in the ultrasonic diagnostic device according to the third embodiment, weighted addition is performed between the arrival time in the path going through the maximum refraction point M and the arrival time in the path going through the non-refraction point S, and the reception time is approximately calculated. Therefore, calculation processing of the reception time can be simplified and an operation time can be significantly reduced. Therefore, the precision of reception beamforming can be improved, and the spatial resolution and the signal S/N ratio can be improved, without increasing the operation amount.

<<Effect by Acoustic Lens Correction>>

Hereinafter, the effects of the embodiment will be described by comparing the quality of ultrasonic images between the reception beamforming according to the first embodiment and reception beamforming without performing acoustic lens correction as a comparative example.

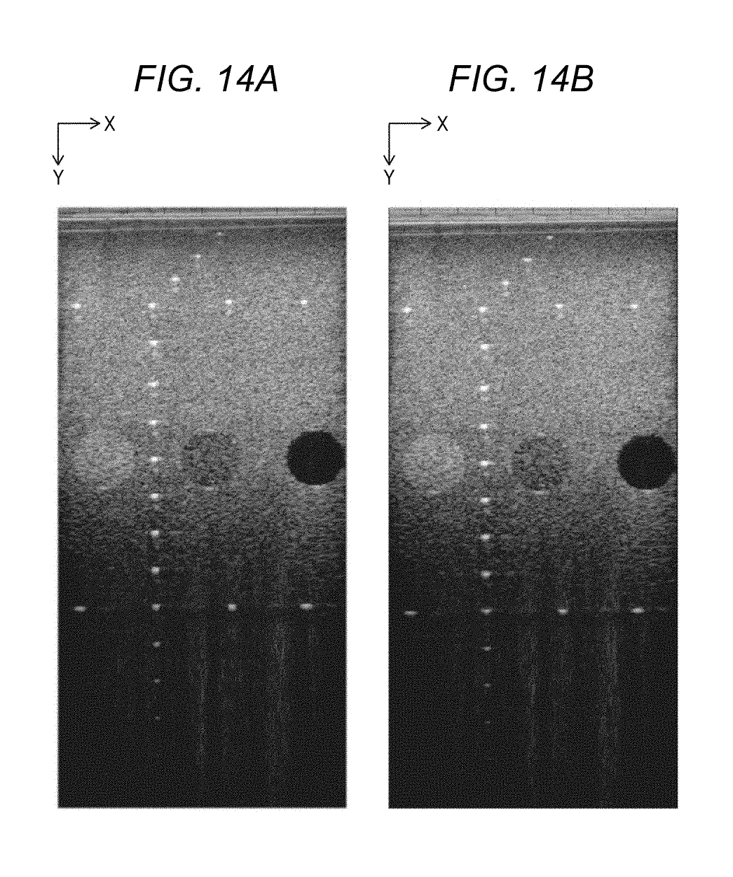

FIGS. 14A and 14B illustrate ultrasonic images (B-mode tomographic images) obtained by imaging the same simulated subject (phantom) by the reception beamforming of an example and first to third comparative examples. FIG. 14A is an example according to the first embodiment, and FIG. 14B corresponds to a comparative example. In the example, the reception beamforming according to the above-described first embodiment is performed. In contrast, in the comparative example, in the reception beamforming, a reception time obtained by dividing a geometrical linear distance between an observation point Pij and a reception transducer Rk by a sound velocity in a subject, without considering an acoustic lens, is used (that is, t₂ in the third embodiment is used as the reception time).

As illustrated in FIG. 14B, in the comparative example, bright spots supposed to be circular bleed in a direction in which transducers are aligned, in particular, in a shallow portion (a region with a small Y coordinate on the upper side in the page). In contrast, as shown in FIG. 14A, the degree of bleeding in a shallow portion is low in the example. This is because, when considering paths between the reception transducers Rk located at both ends of the reception aperture Rx and the observation point Pij, the emission angle θ₁ and the incident angle θ₂ with respect to the refractive surface (the surface of the acoustic lens) become larger as the observation point Pij is shallower, and thus a gap in the reception time due to not considering the acoustic lens becomes large. That is, the reception focus becomes out-of-focus unless the acoustic lens is taken into account as the observation point Pij becomes shallower and the reception aperture Rx becomes wider, and therefore influence on the resolution and the S/N ratio becomes large. In contrast, in the example, such adverse effect due to the acoustic lens can be eliminated.

### Other Modifications According to Embodiments

(1) As the search range of the candidate via point Qₘ, the line segment MS in the first and second embodiments and the arc MS in the first and second modifications have been set with reference to the maximum refraction point M and the non-refraction point S. However, it is sufficient that the search range of the candidate via point Qₘ is based on at least the maximum refraction point M. For example, in the first or second embodiment, an arbitrary line segment MT including the line segment MS (a point T is a point on a half line MS) may be employed as the search range of the candidate via point Qₘ. By doing this, there is no need to specify the non-refraction point S.

Further, in the first embodiment and the first modification, in the case where the sign of the evaluation function J is positive, the point separated by Sₘ from the candidate via point Qₘ₋₁ toward the non-refraction point S side (in the positive direction of x or θ) has been set as the candidate via point Qₘ₊₁. However, a point separated by Sₘ from the candidate via point Qₘ toward the maximum refraction point M side (in the negative direction of x or θ) may be set as the candidate via point Qₘ₊₁.

(2) In the first embodiment and the first modification, the search for the candidate via point Qₘ has been repeated until the refraction point Qt at which the absolute value |J| of the evaluation function J falls below the predetermined threshold value δ is specified. However, for example, an upper limit may be set in advance to the number of searches m of the refraction point Qt, and a point at which the absolute value |J| of the evaluation function J becomes minimum may be employed as the refraction point Qt in the case where the absolute value |J| of the evaluation function J does not fall below the predetermined threshold value δ. For example, in the case of searching for the candidate via point Qₘ with reference to the maximum refraction point M and the non-refraction point S, a point regarded as the refraction point Qt can be specified with the precision of 1/32 the length of the line segment MS (or the arc MS), where the upper limit of m is 5.

(3) In each of the embodiments and modifications, the reception time considering the acoustic lens has been calculated for the reception beamforming. However, by similar calculation, the transmission time considering the acoustic lens may be calculated in the transmission beamforming. Alternatively, the transmission beamforming may be performed on the basis of the calculated transmission time.

(4) In each of the embodiments and modifications, the reception beamforming processing has been performed in synchronization with the transmission of the ultrasonic wave. However, the present invention is not limited to the case. For example, the present invention may be applied in a synthetic aperture method, and the phasing addition may be performed after completion of a plurality of times of ultrasonic transmission/reception of one frame. Further, each of the operations other than the calculation of the reception time is not limited to the above-described case, and arbitrary control may be performed. Further, in each of the embodiments and modifications, the ultrasonic image generator 105 has generated the B-mode image from the acoustic line signal. However, for example, the ultrasonic image generator 105 may perform color flow mapping or shear wave analysis.

(5) In each of the embodiments and modifications, the ultrasonic probe has been the linear probe or the convex probe in which the transducers are concentrically arranged. However, the content of the present disclosure may be applied to an ultrasonic probe having an arbitrary shape by performing appropriate change according to the arrangement form of the transducers.

(6) Note that although the present invention has been described on the basis of the above embodiments, the present invention is not limited to the above embodiments, and the following cases are also included in the present invention.

For example, the present invention may be a computer system including a microprocessor and a memory, and the memory may store the computer program and the microprocessor may be operated according to the computer program. For example, the present invention may include a computer program of the ultrasonic signal processing method of the present invention, and may be a computer system operated according to the computer program (or instructing operations to connected parts).

In addition, a configuration in which the entire or part of the ultrasonic diagnostic device, or the entire or part of the ultrasonic signal processor is constituted by a computer system configured by a microprocessor, a recording medium such a ROM or a RAM, a hard disk unit, and the like is also included in the present invention. In the RAM or the hard disk unit, a computer program for achieving the same operations as the above devices is stored. The devices achieve the functions as the microprocessor is operated according to the computer program.

Further, a part or all of the constituent elements constituting the above devices may be configured by one system, large scale integration (LSI). The system LSI is a super multifunctional LSI manufactured by integrating a plurality of constituent parts on one chip, and is specifically a computer system including a microprocessor, a ROM, a RAM, and the like. These constituent elements may be separately formed into one chip, or may be integrated into one chip to include a part or all of them. Note that the LSI may be referred to as an IC, a system LSI, a super LSI, or an ultra LSI depending on the degree of integration. In the RAM, a computer program for achieving the same operations as the above devices is stored. The system LSI achieves the function as the microprocessor is operated according to the computer program. For example, a case in which the beamforming method of the present invention is stored as a program of the LSI, the LSI is inserted in the computer, and the computer performs a predetermined program (beamforming method) is also included in the present invention.

Note that the technique of integrated circuit is not limited to LSI, and may be realized by a dedicated circuit or a general-purpose processor. After LSI fabrication, a field programmable gate array (FPGA) that can be programmed or a reconfigurable processor that can reconfigure connection and setting of circuit cells inside the LSI may be used.

Furthermore, if an integrated circuit technology replacing the LSI appears due to advancement in the semiconductor technology or another derivative technology, functional block integration may be performed using that technology.

Further, a part or all of the functions of the ultrasonic diagnostic device according to the embodiments may be realized by execution of a program by a processor such as a CPU. A non-transitory computer-readable recording medium in which a program for performing a diagnostic method of the ultrasonic diagnostic device or a beamforming method is recorded may be employed. The program may be performed by another independent computer system by recording and transmitting the program and signals on a recording medium. It is needless to say that the program can be distributed via a transmission medium such as the Internet.

The ultrasonic diagnostic device according to the above embodiment includes the data storage as a storage device. However, the storage device is not limited to the data storage, and may be configured such that a semiconductor memory, a hard disk drive, an optical disk drive, a magnetic storage device, or the like is externally connected to the ultrasonic diagnostic device.

Further, the division of the functional blocks in the block diagram is merely an example. A plurality of functional blocks may be realized as one functional block, one functional block may be divided into a plurality of functional blocks, or some functions may be transferred to another functional block. Further, the functions of a plurality of functional blocks having similar functions may be processed by single hardware or software in parallel or in time division.

Further, the order to execute the above steps is for illustrative purposes to specifically describe the present invention, and an order other than the above may be employed. Further, a part of the above steps may be executed simultaneously (in parallel) with another step.

Further, the probe and the display are externally connected to the ultrasonic diagnostic device. However, the probe and the display may be integrally provided in the ultrasonic diagnostic device.

Further, in the above embodiment, the probe has a probe configuration in which a plurality of piezoelectric elements is arranged in a one-dimensional direction. However, the configuration of the probe is not limited to the embodiment. For example, a two-dimensional array transducer in which a plurality of piezoelectric transducer elements is arrayed in a two-dimensional direction, or a swing probe that mechanically swings a plurality of transducers arrayed in a one-dimensional direction to obtain a three-dimensional tomographic image may be used and can be appropriately used depending on measurement. For example, in the case of using the probe in which piezoelectric transducer elements are two-dimensionally arrayed, irradiation position and direction of an ultrasonic beam to be transmitted can be controlled by individually changing timing to apply a voltage to the piezoelectric transducer element and the value of the voltage.

Further, the probe may include a part of the functions of the transmission/reception unit therein. For example, a transmission electrical signal is generated in the probe, and the transmission electrical signal is converted into an ultrasonic wave, on the basis of a control signal for generating a transmission electrical signal output from the transmission/reception unit. In addition, a configuration to convert a received reflected ultrasonic wave into a reception electrical signal and to generate a reception signal on the basis of the reception electrical signal in the probe can be employed.

Further, at least a part of the functions of the ultrasonic diagnostic device according to the embodiments and its modifications may be combined. Further, the above-used numbers are all for illustrative purposes for specifically describing the present invention, and the present invention is not limited to the exemplified numbers. Further, various modifications obtained by applying changes conceived by those skilled in the art to the present embodiments are also included in the present invention.

<<Conclusion>>

(1) An ultrasonic signal processor according to an embodiment is an ultrasonic signal processor that transmits and receives an ultrasonic wave to and from a subject by joining an ultrasonic probe including a plurality of transducers and an acoustic lens to the subject and generates an acoustic line signal on the basis of a reflected ultrasonic wave, the ultrasonic signal processor including a transmitter that transmits a transmission ultrasonic wave into the subject, using the ultrasonic probe, a receiver that generates a reception signal sequence corresponding to each transducer on the basis of the reflected ultrasonic wave from the subject received by the ultrasonic probe, and a phasing adder that phases and adds, with respect to a plurality of observation points in the subject, the reception signal sequences to generate an acoustic line signal, wherein the phasing adder includes a reception time calculator that calculates, for each observation point and for each transducer, a reception time to when the reflected ultrasonic wave reaches the transducer from the observation point, an ultrasonic velocity in the acoustic lens is slower than an ultrasonic velocity in a region of the subject, the region being in contact with the acoustic lens, and the reception time calculator calculates the reception time to when the ultrasonic wave is propagated from the observation point to the transducer, using a maximum refraction point most adjacent to the transducer on a refractive surface that is a boundary surface between the acoustic lens and the subject.

Further, an ultrasonic signal processing method according to an embodiment is an ultrasonic signal processing method of transmitting and receiving an ultrasonic wave to and from a subject by joining an ultrasonic probe including a plurality of transducers and an acoustic lens to the subject and generating an acoustic line signal on the basis of a reflected ultrasonic wave, the ultrasonic signal processing method including transmitting a transmission ultrasonic wave into the subject, using the ultrasonic probe, generating a reception signal sequence corresponding to each transducer on the basis of the reflected ultrasonic wave from the subject received by the ultrasonic probe, and with respect to a plurality of observation points in the subject, phasing and adding the reception signal sequences to generate an acoustic line signal, wherein a reception time to when the reflected ultrasonic wave reaches the transducer from the observation point is calculated for each observation point and for each transducer, in the phasing and adding, an ultrasonic velocity in the acoustic lens is slower than an ultrasonic velocity in a region of the subject, the region being in contact with the acoustic lens, and the reception time that is a minimum value of time necessary for the ultrasonic wave to propagate from the observation point to the transducer is calculated using a maximum refraction point most adjacent to the transducer on a refractive surface that is a boundary surface between the acoustic lens and the subject, in the calculation of the reception time.

According to the ultrasonic signal processor according to one aspect of the present invention and an ultrasonic diagnostic device using the ultrasonic signal processor, the calculation accuracy of the reception time can be improved for each observation point and each transducer without depending on correction value data. Therefore, an S/N ratio and a spatial resolution of the obtained acoustic line signal can be improved in reception beamforming.

(2) Further the ultrasonic signal processor of (1) may be configured such that the reception time calculator sets a plurality of candidate via points including the maximum refraction point on the refractive surface, regarding each of the candidate via points, an incident angle and an emission angle of the ultrasonic wave with respect to the refractive surface in a path going from the observation point through the candidate via point to reach the transducer are calculated, and specifies a via observation point corresponding to a relationship between the incident angle and the emission angle, the relationship being close to a relationship between an incident angle and an emission angle to be satisfied from a propagation velocity ratio of an ultrasonic wave between a side of the observation point with respect to the refractive surface and a side of the observation point with respect to the refractive surface, and calculates the reception time on the basis of a path going from the observation point through the via observation point to the transducer.

With the above configuration, the reception time based on the path that satisfies the Snell's law can be calculated with high precision.

(3) Further, the ultrasonic signal processor of (2) may be configured such that the reception time calculator sets a point on the refractive surface, the point being separated by a predetermined distance from the maximum refraction point toward a side of a straight line connecting the observation point and the transducer, as a second candidate via point, when the maximum refraction point is a first candidate via point, and sets a point on the refractive surface, the point being separated by ½ of a distance between an n-th candidate via point (n is an integer of 2 or more) and an (n−1)th candidate via point from the n-th candidate via point in a case where the incident angle on a path going through the n-th candidate via point is excessive, or from the (n−1)th candidate via point in a case where the incident angle on a path going through the n-th candidate via point is too small, toward the side of a straight line connecting the observation point and the transducer, as an (n+1)th candidate via point.

With the above configuration, the propagation path of the reflected ultrasonic wave from the observation point to the transducer can be specified with a small number of trials. Therefore, the reception time can be calculated with high precision by a small-scale operation.

(4) Further, the ultrasonic signal processor of (1) may be configured such that the reception time calculator sets a plurality of candidate via points including the maximum refraction point on the refractive surface, and calculates, for each of the candidate via points, a propagation required time of the ultrasonic wave on the path going from the observation point through the candidate via point to reach the transducer, and calculates a smallest value, of a plurality of the propagation required times, as the reception time.

With the above configuration, the reception time can be directly calculated without specifying the propagation path of the reflected ultrasonic wave from the observation point to the transducer.

(5) Further, the ultrasonic signal processor of (1) may be configured such that the reception time calculator specifies an intersection point between the refractive surface and a straight line connecting the observation point and the transducer, as a path calculation point, calculates a first time, using a path from the observation point to at least one of the maximum refraction point and the path calculation point, calculates a second time, using a path from at least one of the maximum refraction point and the path calculation point to the transducer and calculates the reception time, using the first time and the second time.

With the above configuration, the reception time can be calculated by approximate calculation with a small operation amount without specifying the propagation path of the reflected ultrasonic wave from the observation point to the transducer.

(6) Further, the ultrasonic signal processor of (5) may be configured such that the reception time calculator calculates the first time by linear combination of a time to when the ultrasonic wave passes through the path from the observation point to the maximum refraction point and a time to when the ultrasonic wave passes through the path from the observation point to the path calculation point.

(7) Further, the ultrasonic signal processor of (5) or (6) may be configured such that the reception time calculator calculates the second time by linear combination of a time to when the ultrasonic wave passes through the path from the maximum refraction point to the transducer and a time to when the ultrasonic wave passes through the path from the path calculation point to the transducer.

With these configurations, the precision of approximation can be further improved by performing approximate calculation for each of the inside of the subject and the inside of the acoustic lens.

The ultrasonic signal processor, the ultrasonic diagnostic device, the ultrasonic signal processing method, the program, and the computer-readable non-transitory recording medium according to the present disclosure are useful in improvement of performance, in particular, the resolution and the S/N ratio, in the case of using the ultrasonic probe including the acoustic lens.

Although embodiments of the present invention have been described and illustrated in detail, the disclosed embodiments are made for purposes of illustration and example only and not limitation. The scope of the present invention should be interpreted by terms of the appended claims.

What is claimed is:

1. An ultrasonic signal processor that transmits and receives an ultrasonic wave to and from a subject by joining an ultrasonic probe including a plurality of transducers and an acoustic lens to the subject and generates an acoustic line signal on the basis of a reflected ultrasonic wave, the ultrasonic signal processor comprising:
a transmitter that transmits a transmission ultrasonic wave into the subject, using the ultrasonic probe;
a receiver that generates a reception signal sequence corresponding to each transducer on the basis of the reflected ultrasonic wave from the subject received by the ultrasonic probe; and
a phasing adder that phases and adds, with respect to a plurality of observation points in the subject, the reception signal sequences to generate an acoustic line signal, wherein
the phasing adder includes a reception time calculator that calculates, for each observation point and for each transducer, a reception time to when the reflected ultrasonic wave reaches the transducer from the observation point,
an ultrasonic velocity in the acoustic lens is slower than an ultrasonic velocity in a region of the subject, the region being in contact with the acoustic lens, and
the reception time calculator calculates the reception time to when the ultrasonic wave is propagated from the observation point to the transducer, using a maximum refraction point most adjacent to the transducer on a refractive surface that is a boundary surface between the acoustic lens and the subject.

2. The ultrasonic signal processor according to claim 1, wherein
the reception time calculator sets a plurality of candidate via points including the maximum refraction point on the refractive surface,
regarding each of the candidate via points, an incident angle and an emission angle of the ultrasonic wave with respect to the refractive surface in a path going from the observation point through the candidate via point to reach the transducer are calculated, and
specifies a via observation point corresponding to a relationship between the incident angle and the emission angle, the relationship being close to a relationship between an incident angle and an emission angle to be satisfied from a propagation velocity ratio of an ultrasonic wave between a side of the observation point with respect to the refractive surface and a side of the observation point with respect to the refractive surface, and calculates the reception time on the basis of a path going from the observation point through the via observation point to tie transducer.

3. The ultrasonic signal processor according to claim 2, wherein
the reception time calculator sets a point on the refractive surface, the point being separated by a predetermined distance from the maximum refraction point toward a side of a straight line connecting the observation point and the transducer, as a second candidate via point, when the maximum refraction point is a first candidate via point, and
sets a point on the refractive surface, the point being separated by ½ of a distance between an n-th candidate via point (n is an integer of 2 or more) and an (n−1)th candidate via point from the n-th candidate via point in a case where the incident angle on a path going through the n-th candidate via point is excessive, or from the (n−1)th candidate via point in a case where the incident angle on a path going through the n-th candidate via point is too small, toward the side of a straight line connecting the observation point and the transducer, as an (n+1)th candidate via point.

4. The ultrasonic signal processor according to claim 1, wherein
the reception time calculator sets a plurality of candidate via points including the maximum refraction point on the refractive surface, and
calculates, for each of the candidate via points, a propagation required time of the ultrasonic wave on the path going from the observation point through the candidate via point to reach the transducer, and
calculates a smallest value, of a plurality of the propagation required times, as the reception time.

5. The ultrasonic signal processor according to claim 1, wherein
the reception time calculator specifies an intersection point between the refractive surface and a straight line connecting the observation point and the transducer, as a path calculation point,
calculates a first time, using a path from the observation point to at least one of the maximum refraction point and the path calculation point,
calculates a second time, using a path from at least one of the maximum refraction point and the path calculation point to the transducer, and
calculates the reception time, using the first time and the second time.

6. The ultrasonic signal processor according to claim 5, wherein
the reception time calculator calculates the first time by linear combination of a time to when the ultrasonic wave passes through the path from the observation point to the maximum refraction point and a time to when the ultrasonic wave passes through the path from the observation point to the path calculation point.

7. The ultrasonic signal processor according to claim 5, wherein
the reception time calculator calculates the second time by linear combination of a time to when the ultrasonic wave passes through the path from the maximum refraction point to the transducer and a time to when the ultrasonic wave passes through the path from the path calculation point to the transducer.

8. An ultrasonic diagnostic device comprising:
an ultrasonic probe including an acoustic lens; and
the ultrasonic signal processor according to claim 1.

9. An ultrasonic signal processing method of transmitting and receiving an ultrasonic wave to and from a subject by joining an ultrasonic probe including a plurality of transducers and an acoustic lens to the subject and generating an acoustic line signal on the basis of a reflected ultrasonic wave, the ultrasonic signal processing method comprising:
transmitting a transmission ultrasonic wave into the subject, using the ultrasonic probe;
generating a reception signal sequence corresponding to each transducer on the basis of the reflected ultrasonic wave from the subject received by the ultrasonic probe; and
with respect to a plurality of observation points in the subject, phasing and adding the reception signal sequences to generate an acoustic line signal, wherein
a reception time to when the reflected ultrasonic wave reaches the transducer from the observation point is calculated for each observation point and for each transducer, in the phasing and adding,
an ultrasonic velocity in the acoustic lens is slower than an ultrasonic velocity in a region of the subject, the region being in contact with the acoustic lens, and
the reception time that is a minimum value of time necessary for the ultrasonic wave to propagate from the observation point to the transducer is calculated using a maximum refraction point most adjacent to the transducer on a refractive surface that is a boundary surface between the acoustic lens and the subject, in the calculation of the reception time.
